(19) United States
(12) Patent Application Publication
(10) Pub. No.: US 2007/0191353 A1
(43) Pub. Date: Aug. 16, 2007

(54) Use of thiadiazoleurea derivatives

(76) Inventors: Lars Burgdorf, Frankfurt (DE); Frank Stieber, Heidelberg (DE); Hans-Peter Buchstaller, Griesheim (AT); Soheila Anzali, Seeheim-Jugenheim (DE); Christiane Amendt, Darmstadt (DE); Hartmut Greiner, Weiterstadt (DE); Matthias Grell, Darmstadt (DE); Christian Sirrenberg, Darmstadt (DE); Frank Zenke, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/590,729

(22) PCT Filed: Jan. 31, 2005
(86) PCT No.: PCT/EP05/00908

§ 371 Date: Aug. 25, 2006

(30) Foreign Application Priority Data
Feb. 26, 2004 (DE) 10 2004 009933.2

(51) Int. Cl. A61K 31/541; A61K 31/5377; A61K 31/496; A61K 31/454; A61K 31/433; C07D 417/02

(52) U.S. Cl. 514/227.5; 514/235.5; 514/254.03; 514/326; 514/362; 544/60; 548/138; 544/134; 544/367; 546/209

(57) ABSTRACT

Use of compounds of the formula (I), in which Ar¹, Ar² and Z have the meanings indicated in Patent Claim 1, for the prophylaxis and/or treatment of diseases in which the inhibition, regulation and/or modulation of signal transduction by kinases, in particular RAF kinases, plays a role.

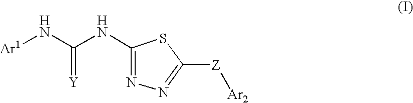

The present invention relates to compounds and the use of compounds in which the inhibition, regulation and/or modulation of signal transduction by kinases, in particular serine/threonine and/or tyrosine kinases, plays a role, furthermore pharmaceutical compositions which comprise these compounds, and the use of the compounds for the treatment of kinase-induced diseases.

The present invention relates, in particular, to the use of the compounds of the formula I for the preparation of a medicament for the prophylaxis and/or treatment of diseases, in particular tumours and/or diseases which are caused, mediated and/or propagated by angiogenesis. Compounds of the formula I are effective inhibitors of tyrosine kinases, in particular TIE-2 and VEGFR, and of Raf kinases.

It has been found that the compounds of the formula I are capable of inhibiting, regulating and/or modulating signal transduction mediated by kinases, in particular by tyrosine kinases and/or Raf kinases. In particular, the compounds according to the invention are suitable as inhibitors of tyrosine kinases and/or Raf kinases. Thus, the compounds of the formula I can be employed for the preparation of medicaments for the prophylaxis and/or treatment of diseases that are caused, mediated and/or propagated by kinases and/or by kinase-mediated signal transduction or by angiogenesis. Thus, the compounds according to the invention are suitable for the treatment and/or prophylaxis of cancer, tumour growth, arteriosclerosis, age-induced macular degeneration, diabetic retinopathy, inflammatory diseases and the like in mammals.

Tyrosine kinases are a class of enzymes which catalyse the transfer of the terminal phosphate of adenosine triphosphate to tyrosine residues in protein substrates. It is thought that tyrosine kinases, through substrate phosphorylation, play a crucial role in signal transduction for a number of cellular functions. Although the precise mechanisms of signal transduction are still unclear, tyrosine kinases have been shown to be important factors in cell proliferation, carcinogenesis and cell differentiation.

Tyrosine kinases can be categorised as receptor-type tyrosine kinases or non-receptor-type tyrosine kinases. Receptor-type tyrosine kinases have an extracellular portion, a transmembrane portion and an intracellular portion, while non-receptor-type tyrosine kinases are exclusively intracellular.

Receptor-type tyrosine kinases consist of a multiplicity of transmembrane receptors with different biological activity. Thus, about 20 different subfamilies of receptor-type tyrosine kinases have been identified. One tyrosine kinase subfamily, known as the EGFR or HER subfamily, consists of EGFR, HER2, HER3 and HER4. Ligands from this subfamily of receptors include epithelial growth factor (EGF), tissue growth factor (TGF-α), amphiregulin, HB-EGF, betacellulin and heregulin. Another subfamily of these receptor-type tyrosine kinases is the insulin subfamily, which includes INS-R, IGF-IR and IR-R. The PDGF subfamily includes the PDGF-α and -β receptor, CSFIR, c-kit and FLK-II. In addition, there is the FLK family, which consists of the kinase insert domain receptor (KDR) or VEGFR-2, foetal liver kinase-1 (FLK-1), foetal liver kinase-4 (FLK-4) and fms tyrosine kinase-1 (flt-1) or VEGFR-1. The PDGF and FLK family are usually combined in the group of the split kinase domain receptor tyrosine kinases (Laird, A. D. and J. M. Cherrington, Expert. Opin. Investig. Drugs 12(1): 51-64, 2003) due to the similarities between the two groups. For a detailed discussion of receptor-type tyrosine kinases, see the paper by Plowman et al., DN & P 7(6):334-339, 1994, which is incorporated herein by way of reference.

Non-receptor-type tyrosine kinases likewise consist of a multiplicity of subfamilies, including Src, Frk, Btk, Csk, Abl, Zap70, Fes/Fps, Fak, Jak, Ack, and LIMK. Each of these subfamilies is further sub-divided into different sub-groups. For example, the Src subfamily is one of the largest subfamilies. It includes Src, Yes, Fyn, Lyn, Lck, Blk, Hck, Fgr and Yrk. The Src subfamily of enzymes has been linked to oncogenesis. For a more detailed discussion of non-receptor-type tyrosine kinases, see the paper by Bolen, Oncogene, 8:2025-2031 (1993), which is incorporated herein by way of reference.

Both receptor-type tyrosine kinases and non-receptor-type tyrosine kinases are involved in cellular signalling pathways leading to conditions such as cancer, psoriasis and hyperimmune responses.

Cancer is a disease whose causes are to be seen in disturbed signal transduction. In particular, deregulated signal transduction via tyrosine kinases plays a major role in the growth and spread of cancer (Blume-Jensen, P. and T. Hunter, Nature 411: 355-365, 2001; Hanahan D. and R. A. Weinberg, Cell 100:57-70, 2000). Tyrosine kinases and in particular receptor-type tyrosine kinases and the growth factors binding to them may thus be involved in deregulated apoptosis, tissue invasion, metastasis and generally in signal transduction mechanisms which lead to cancer.

In particular, receptor-type tyrosine kinases play a role in angiogenesis, a further important mechanism in the growth and spread of cancer (Mustonen and Alitalo, J. Cell Biol. 129:895-898, 1995). One of these receptor-type tyrosine kinases is foetal liver kinase 1, also referred to as FLK-1. The human analogue of FLK-1 is the kinase insert domain-containing receptor KDR, which is also known as vascular endothelial cell growth factor receptor 2 or VEGFR-2, since it binds VEGF with high affinity. The murine version of this receptor has been called NYK (Oelrichs et al., Oncogene 8(1):11-15, 1993). VEGF and KDR are a ligand-receptor pair which plays a vital role in the proliferation of vascular endothelial cells and the formation and sprouting of blood vessels, referred to as vasculogenesis and angiogenesis respectively.

Angiogenesis is characterised by excessive activity of vascular endothelial growth factor (VEGF). VEGF actually consists of a family of ligands (Klagsburn and D'Amore, Cytokine & Growth Factor Reviews 7:259-270, 1996). VEGF binds the high-affinity membrane-spanning tyrosine kinase receptor KDR and the related fms tyrosine kinase-1, also known as Flt-1 or vascular endothelial cell growth factor receptor 1 (VEGFR-1). Cell culture and gene knockout experiments indicate that each receptor contributes to different aspects of angiogenesis. KDR mediates the mitogenic function of VEGF, whereas Flt-1 appears to modulate non-mitogenic functions, such as those associated with cellular adhesion. Inhibiting KDR thus modulates the level of mitogenic VEGF activity. In fact, tumour growth has been shown to be influenced by the antiangiogenic effect of VEGF receptor antagonists (Kim et al., Nature 362, pp. 841-844, 1993).

Expression of VEGF is also significantly increased in hypoxic regions of animal and human tumours adjacent to areas of necrosis. In addition, VEGF is upregulated by the expression of the oncogenes ras, raf, src and p53 mutants (all of which are of importance in combating cancer). Anti-VEGF monoclonal antibodies inhibit the growth of human tumours in nude mice. The same tumour cells continue to express VEGF in culture, but here the antibodies do not diminish the mitotic rate, i.e. the tumour-derived VEGF does not function as an autocrine mitogenic factor. VEGF instead contributes to tumour growth in vivo by promoting angiogenesis through its paracrine vascular endothelial cell chemotactic and mitogenic activity. The monoclonal anti-VEGF antibodies also inhibit the growth of typically less well vascularised human colon carcinomas in athymic mice and decrease the number of tumours arising from inoculated cells.

Solid tumours can be treated with tyrosine kinase inhibitors since these tumours depend on angiogenesis for the formation of the blood vessels that are necessary to support their growth. These solid tumours include monocytic leukaemia, carcinoma of the brain, urogenital tract, lymphatic system, stomach, larynx and lung, including lung adenocarcinoma and small cell lung carcinoma.

Further examples of solid tumours include carcinomas in which overexpression or activation of Raf-activating oncogenes (for example K-ras, erb-B) is observed. These carcinomas include pancreatic and breast carcinoma. Inhibitors of these tyrosine kinases and/or Raf kinases are therefore suitable for the prevention and treatment of proliferative diseases caused by these enzymes.

The angiogenic activity of VEGF is not limited to tumours. VEGF is also responsible for the angiogenic activity produced in or near the retina in diabetic retinopathy. This vascular growth in the retina leads to visual degeneration culminating in blindness. Ocular VEGF mRNA and protein levels that lead to neovascularisation are further elevated by conditions such as retinal vein occlusion in primates and decreased pO₂ level in mice. Intraocular injections of anti-VEGF monoclonal antibodies or VEGF receptor immunofusions inhibit ocular neovascularisation in both primate and rodent models. Irrespective of the cause of induction of VEGF in human diabetic retinopathy, inhibition of the VEGF in the eye is suitable for treating this disease.

The expression of a VEGF-binding construct of Flk-1, Flt-1, the mouse KDR receptor homologue truncated to eliminate the cytoplasmic tyrosine kinase domains but retaining a membrane anchor, in viruses virtually stops the growth of a transplantable glioblastoma in mice, presumably by the dominant negative mechanism of heterodimer formation with membrane-spanning endothelial cell VEGF receptors. Embryonic stem cells, which normally grow as solid tumours in nude mice, do not form detectable tumours if both VEGF alleles are knocked out. Taken together, these data indicate the role of VEGF in the growth of solid tumours. Inhibition of KDR or Flt-1 is involved in pathological angiogenesis, and inhibitors of these receptors are suitable for the treatment of diseases in which angiogenesis is part of the overall pathology, for example inflammation, diabetic retinal vascularisation, as well as various forms of cancer, since tumour growth is known to be dependent on angiogenesis (Weidner et al., N. Engl. J. Med., 324, pp. 1-8, 1991).

The present invention is directed to the use of compounds of the formula I which are capable of regulating, modulating or inhibiting VEGFR for the prevention and/or treatment of diseases in connection with unregulated or disturbed VEGFR activity. In particular, the compounds can therefore be employed in the treatment of certain forms of cancer and in the case of diseases caused by pathological angiogenesis, such as diabetic retinopathy or inflammation.

Furthermore, compounds of the formula I can be used for the isolation and investigation of the activity or expression of VEGFR. In addition, they are particularly suitable for use in diagnostic methods for diseases in connection with unregulated or disturbed VEGFR activity.

Angiopoietin 1 (Ang1), a ligand for the endothelium-specific receptor-type tyrosine kinase TIE-2, is a novel angiogenic factor (Davis et al, Cell, 1996, 87:1161-1169; Partanen et al, Mol. Cell Biol., 12:1698-1707 (1992); U.S. Pat. Nos. 5,521,073; 5,879,672; 5,877,020; and 6,030,831). The acronym TIE stands for “tyrosine kinase with Ig and EGF homology domains”. TIE is used for the identification of a class of receptor-type tyrosine kinases which are expressed exclusively in vascular endothelial cells and early haemopoietic cells. TIE receptor kinases are typically characterised by the presence of an EGF-like domain and an immunoglobulin (IG)-like domain which consists of extracellular fold units stabilised by disulfide bridge bonds between the chains (Partanen et al., Curr. Topics Microbiol. Immunol., 1999, 237: 159-172). In contrast to VEGF, which exerts its function during the early stages of vascular development, Ang1 and its receptor TIE-2 act during the later stages of vascular development, i.e. during vascular transformation (transformation relates to the formation of a vascular lumen) and maturing (Yancopoulos et al., Cell, 1998, 93:661-664; Peters, K. G., Circ. Res., 1998, 83(3):342-3; Suri et al., Cell 87, 1171-1180 (1996)).

Accordingly, it would be expected that inhibition of TIE-2 should interrupt the transformation and maturing of a new vascular system initiated by angiogenesis and should thus interrupt the angiogenesis process. Furthermore, inhibition at the kinase domain binding site of VEGFR-2 would block phosphorylation of tyrosine residues and serve to interrupt initiation of angiogenesis. It must therefore be assumed that inhibition of TIE-2 and/or VEGFR-2 should prevent tumour angiogenesis and serve to slow or completely eliminate tumour growth.

Accordingly, treatment of cancer and other diseases associated with inappropriate angiogenesis could be provided with inhibitors of TIE-2 and/or VEGFR-2.

The compounds of the formula I are capable of inhibiting, regulating and/or modulating TIE-2 and are thus suitable for the prevention and/or treatment of diseases in connection with unregulated or disturbed TIE-2 activity. In particular, the compounds can therefore be used for the preparation of medicaments for the prophylaxis and/or treatment of certain forms of cancer and in the case of diseases caused by pathological angiogenesis, such as diabetic retinopathy or inflammation.

Furthermore, the compounds of the formula I can be used for the isolation and investigation of the activity or expression of TIE-2. In addition, they are particularly suitable for use in diagnostic methods for diseases in connection with unregulated or disturbed TIE-2 activity.

The compounds according to the invention can furthermore be used in order to provide additive or synergistic effects in certain existing cancer chemotherapies and radiotherapies, and/or can be used to restore the efficacy of certain existing cancer chemotherapies and radiotherapies.

The present invention furthermore relates to the use of the compounds of the formula I for the inhibition of Raf kinases.

Protein phosphorylation is a fundamental process for the regulation of cellular functions. The coordinated action of both protein kinases and phosphatases controls the degrees of phosphorylation and, hence, the activity of specific target proteins. One of the predominant roles of protein phosphorylation is in signal transduction, where extracellular signals are amplified and propagated by a cascade of protein phosphorylation and dephosphorylation events, for example in the p21^{ras}/raf pathway.

The p21^{ras} gene was discovered as an oncogene of the Harvey (H-Ras) and Kirsten (K-Ras) rat sarcoma viruses. In humans, characteristic mutations in the cellular Ras gene (c-Ras) have been associated with many different types of cancer. These mutant alleles, which render Ras constitutively active, have been shown to transform cells, such as, for example, the murine cell line NIH 3T3, in culture.

The p21^{ras} oncogene is an important contributory factor in the development and progression of human solid carcinomas and is mutated in 30% of all human carcinomas (Bolton et al. (1994) Ann. Rep. Med. Chem., 29, 165-74; Bos. (1989) Cancer Res., 49, 4682-9). In its normal, unmutated form, the Ras protein is a key element of the signal transduction cascade directed by growth factor receptors in almost all tissues (Avruch et al. (1994) Trends Biochem. Sci., 19, 279-83).

Biochemically, Ras is a guanine nucleotide binding protein, and the cycling between a GTP-bound activated and a GDP-bound resting form is strictly controlled by Ras endogenous GTPase activity and other regulatory proteins. The Ras gene product binds to guanine triphosphate (GTP) and guanine diphosphate (GDP) and hydrolyses GTP to GDP. Ras is active in the GTP-bound state. In the Ras mutants in cancer cells, the endogenous GTPase activity is reduced, and the protein consequently transmits constitutive growth signals to downstream effectors, such as, for example, the enzyme Raf kinase. This leads to the cancerous growth of the cells which carry these mutants (Magnuson et al. (1994) Semin. Cancer Biol., 5, 247-53). The Ras proto-oncogene requires a functionally intact C-Raf-1 proto-oncogene in order to transduce growth and differentiation signals initiated by receptor- and non-receptor-type tyrosine kinases in higher eukaryotes.

Activated Ras is necessary for the activation of the C-Raf-1 proto-oncogene, but the biochemical steps through which Ras activates the Raf-1 protein (Ser/Thr) kinase are now well characterised. It has been shown that inhibiting the effect of active Ras by inhibiting the Raf kinase signalling pathway by administration of deactivating antibodies to Raf kinase or by co-expression of dominant negative Raf kinase or dominant negative MEK (MAPKK), the substrate of Raf kinase, leads to reversion of transformed cells to the normal growth phenotype, see: Daum et al. (1994) Trends Biochem. Sci., 19, 474-80; Fridman et al. (1994) J Biol. Chem., 269, 30105-8. Kolch et al. (1991) Nature, 349, 426-28) and to the review Weinstein-Oppenheimer et al. Pharm. & Therap. (2000), 88, 229-279.

Similarly, inhibition of Raf kinase (by antisense oligodeoxynucleotides) has been correlated in vitro and in vivo with inhibition of the growth of a variety of types of human tumour (Monia et al., Nat. Med. 1996, 2, 668-75); Geiger et al. (1997), Clin. Cancer Res. 3(7):1179-85; Lau et al. (2002), Antisense Nucl. Acid. Drug Dev. 12(1): 11-20; Mc Phillips et al. (2001), Br. J. Cancer 85(11): 1754-8)

Raf serine- and threonine-specific protein kinases are non-receptor-type enzymes that stimulate cell growth in a variety of cellular systems (Rapp, U. R., et al. (1988) in The Oncogene Handbook; T. Curran, E. P. Reddy and A. Skalka (eds.) Elsevier Science Publishers; The Netherlands, pp. 213-253; Rapp, U. R., et al. (1988) Cold Spring Harbor Sym. Quant. Biol. 53:173-184; Rapp, U. R., et al. (1990) Inv Curr. Top. Microbiol. Immunol. Potter and Melchers (eds.), Berlin, Springer-Verlag 166:129-139).

Three isozymes have been characterised:

C-Raf (Raf-1) (Bonner, T. I., et al. (1986) Nucleic Acids Res. 14: 1009-1015). A-Raf (Beck, T. W., et al. (1987) Nucleic Acids Res. 15:595-609), and B-Raf (Qkawa, S., et al. (1998) Mol. Cell. Biol. 8: 2651-2654; Sithanandam, G. et al. (1990) Oncogene:1775). These enzymes differ in their expression in various tissues. Raf-1 is expressed in all organs and in all cell lines that have been examined, and A- and B-Raf are expressed in urogenital and brain tissues respectively (Storm, S. M. (1990) Oncogene 5:345-351).

Raf genes are proto-oncogenes: they can initiate malignant transformation of cells when expressed in specifically altered forms. Genetic changes that lead to oncogenic activation generate a constitutively active protein kinase by removal of or interference with an N-terminal negative regulatory domain of the protein (Heidecker, G., et al. (1990) Mol. Cell. Biol. 10:2503-2512; Rapp, U. R., et al. (1987) in Oncogenes and Cancer; S. A. Aaronson, J. Bishop, T. Sugimura, M. Terada, K. Toyoshima and P. K. Vogt (eds.) Japan Scientific Press, Tokyo). Microinjection into NIH 3T3 cells of oncogenically activated, but not wild-type, versions of the Raf protein prepared with Escherichia coli expression vectors results in morphological transformation and stimulates DNA synthesis (Rapp, U. R., et al. (1987) in Oncogenes and Cancer; S. A. Aaronson, J. Bishop, T. Sugimura, M. Terada, K. Toyoshima, and P. K. Vogt (eds.) Japan Scientific Press, Tokyo; Smith, M. R., et al. (1990) Mol. Cell. Biol. 10:3828-3833). Activating mutants of B-Raf have been identified in various types of human cancer, for example of the intenstine, the ovaries, melanomas and sarcomas (Davies, H. et al. (2002), Nature 417, 949-945; published online 9 Jun. 2002, 10.1038/-nature 00766). The predominant mutation is a single phosphomimetic substitution in the kinase-activation domain (V599E), which results in constitutive kinase activity and transformation of NIH3T3 cells.

Consequently, activated Raf-1 is an intracellular activator of cell growth. Raf-1 protein serine kinase is a candidate for the downstream effector of mitogen signal transduction, since Raf oncogenes counter the growth arrest resulting from blockage of cellular Ras activity due either to a cellular mutation (Ras revertant cells) or microinjection of anti-Ras antibodies (Rapp, U. R., et al. (1988) in The Oncogene Handbook, T. Curran, E. P. Reddy and A. Skalka (eds.), Elsevier Science Publishers; The Netherlands, pp. 213-253; Smith, M. R., et al. (1986) Nature (London) 320:540-543).

C-Raf function is required for transformation by a variety of membrane-bound oncogenes and for growth stimulation by mitogens contained in serums (Smith, M. R., et al. (1986) Nature (London) 320:540-543). Raf-1 protein serine kinase activity is regulated by mitogens via phosphorylation (Morrison, D. K., et al. (1989) Cell 58:648-657), which also effects sub-cellular distribution (Olah, Z., et al. (1991) Exp. Brain Res. 84:403; Rapp, U. R., et al. (1988) Cold Spring Harbor Sym. Quant. Biol. 53:173-184. Raf-1-activating growth factors include platelet-derived growth factor (PDGF) (Morrison, D. K., et al. (1988) Proc. Natl. Acad. Sci. USA 85:8855-8859), colony-stimulating factor (Baccarini, M., et al. (1990) EMBO J. 9:3649-3657), insulin (Blackshear, P. J., et al. (1990) J. Biol. Chem. 265: 12115-12118), epidermal growth factor (EGF) (Morrison, R. K., et al. (1988) Proc. Natl. Acad. Sci. USA 85:8855-8859), interleukin-2 (Turner, B. C., et al. (1991) Proc. Natl. Acad. Sci. USA 88:1227) and interleukin-3 and granulocyte macrophage colony-stimulating factor (Carroll, M. P., et al. (1990) J. Biol. Chem. 265:19812-19817).

After mitogen treatment of cells, the transiently activated Raf-1 protein serine kinase translocates to the perinuclear area and the nucleus (Olah, Z., et al. (1991) Exp. Brain Res. 84:403; Rapp, U. R., et al. (1988) Cold Spring Habor Sym. Quant. Biol. 53:173-184). Cells containing activated Raf are altered in their pattern of gene expression (Heidecker, G., et al. (1989) in Genes and signal transduction in multistage carcinogenesis, N. Colburn (ed.), Marcel Dekker, Inc., New York, pp. 339-374) and Raf-oncogenes activate transcription from Ap-I/PEA3-dependent promoters in transient transfection assays (Jamal, S., et al. (1990) Science 344:463-466; Kaibuchi, K., et al. (1989) J. Biol. Chem. 264:20855-20858; Wasylyk, C., et al. (1989) Mol. Cell. Biol. 9:2247-2250).

There are at least two independent pathways for Raf-1 activation by extracellular mitogens: one involving protein kinase C (KC) and a second initiated by protein tyrosine kinases (Blackshear, P. J., et al. (1990) J. Biol. Chem. 265:12131-12134; Kovacina, K. S., et al. (1990) J. Biol. Chem. 265:12115-12118; Morrison, D. K., et al. (1988) Proc. Natl. Acad. Sci. USA 85:8855-8859; Siegel, J. N., et al. (1990) J. Biol. Chem. 265:18472-18480; Turner, B. C., et al. (1991) Proc. Natl. Acad. Sci. USA 88:1227). In each case, activation involves Raf-1 protein phosphorylation. Raf-1 phosphorylation may be a consequence of a kinase cascade amplified by autophosphorylation or may be caused entirely by autophosphorylation initiated by binding of a presumed activating ligand to the Raf-1 regulatory domain, analogous to PKC activation by diacylglycerol (Nishizuka, Y. (1986) Science 233:305-312).

One of the principal mechanisms by which cellular regulation is effected is by the transduction of extracellular signals across the membrane that in turn modulate biochemical pathways within the cell. Protein phosphorylation represents one course by which intracellular signals are propagated from molecule to molecule resulting finally in a cellular response. These signal transduction cascades are highly regulated and often overlap, as is evident from the existence of many protein kinases as well as phosphatases. Phosphorylation of proteins occurs predominantly at serine, threonine or tyrosine residues, and protein kinases have therefore been classified by their specificity of phosphorylation site, i.e. serine/threonine kinases and tyrosine kinases. Since phosphorylation is such a ubiquitous process within cells and since cellular phenotypes are largely influenced by the activity of these pathways, it is currently believed that a number of disease states and/or diseases are attributable to either aberrant activation or functional mutations in the molecular components of kinase cascades. Consequently, considerable attention has been devoted to the characterisation of these proteins and compounds that are able to modulate their activity (for review see: Weinstein-Oppenheimer et al. Pharma. &. Therap., 2000, 88, 229-279).

Surprisingly, it has been found that compounds of the formula I can interact with signalling pathways, particularly the signalling pathways described herein and preferably the Raf kinase signalling pathway. The compounds of the formula I preferably exhibit an advantageous biological activity, which is easily detectable in enzyme-based assays, for example assays as described herein. In enzyme-based assays of this type, the compounds of the formula I preferably exhibit and cause an inhibiting effect, which is usually documented by IC₅₀ values in a suitable range, preferably in the micro-molar range and more preferably in the nanomolar range.

Since the enzyme is a downstream effector of p21^{ras}, the inhibitors prove to be suitable in pharmaceutical compositions for use in human or veterinary medicine where inhibition of the Raf kinase pathway is indicated, for example in the treatment of tumours and/or cancerous cell growth mediated by Raf kinase. In particular, the compounds are suitable in the treatment of human and animal solid cancers, for example murine cancer, since the progression of these cancers is dependent upon the Ras protein signal transduction cascade and therefore responsive to treatment by interruption of the cascade, i.e. by inhibiting Raf kinase. Accordingly, the compounds of the formula I or a pharmaceutically acceptable salt thereof is administered for the treatment of diseases mediated by the Raf kinase pathway, especially cancer, including solid cancers, such as, for example, carcinomas (for example of the lungs, pancreas, thyroid, bladder or colon), myeloid diseases (for example myeloid leukaemia) or adenomas (for example villous colon adenoma), pathological angiogenesis and metastatic cell migration. The compounds are furthermore suitable in the treatment of complement activation dependent chronic inflammation (Niculescu et al. (2002) Immunol. Res., 24:191-199) and HIV-1 (human immunodeficiency virus type 1) induced immunodeficiency (Popik et al. (1998) J Virol, 72: 6406-6413), infectious disease, influenza A virus (Pleschka, S. et al. (2001), Nat. Cell. Biol., 3(3):301-5) and Heliobacter pylori infection (Wessler, S. et al. (2002), FASEB J., 16(3): 417-9).

As discussed herein, these signalling pathways are relevant for various diseases. Accordingly, the compounds of the formula I are useful in the prophylaxis and/or treatment of diseases which are dependent on the said signalling pathways through interaction with one or more of the said signalling pathways.

The present invention therefore relates to the use of one or more of the compounds of the formula I 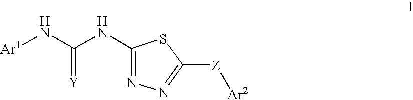
in which
Ar¹ denotes phenyl, naphthyl, biphenyl or Het, each of which is unsubstituted or mono-, di-, tri-, tetra- or pentasubstituted by R¹,
Ar² denotes phenyl, naphthyl, biphenyl or Het, each of which is unsubstituted or mono-, di-, tri-, tetra- or pentasubstituted by R²,
Y denotes O, S, CH—NO₂, C(CN)₂ or N—R⁴,
Z denotes —O—, —S—, —CH₂—(CH₂)ₙ—, —(CH₂)ₙ—CHA-, —CHA-(CH₂)ₙ—, —C(═O)—, —CH(OH)—, —(CHA)ₙO—, —(CH₂)ₙO—, —O(CHA)ₙ—, —O(CH₂)ₙ—, —(CH₂)ₙS—, —S(CH₂)ₙ—, —(CH₂)ₙNH—, —NH(CH₂)ₙ—, —(CH₂)ₙNA-, —NA(CH₂)ₙ—, —CHHal- or —C(Hal)₂-,
Het denotes a mono- or bicyclic aromatic heterocycle having 1 to 4 N, O and/or S atoms,
R¹, R², independently of one another, denote A, Ar′, OR³, SR³, OAr′, SAr′, N(R³)₂, NHAr′, Hal, NO₂, CN, (CH₂)ₙCOOR³, (CH₂)ₙCON(R³)₂, COR³, S(O)ₘA, S(O)ₘAr′, NHCOA, NHCOAr′, NHSOₘA, NHSOₘAr′, SO₂N(R³)₂, O(CH₂)ₙ—N(R³)₂, O(CH₂)ₙNHR³₃, O(CH₂)ₙNA₂, O(CH₂)ₙC(CH₃)₂(CH₂)ₙN(R³)₂, NH(CH₂)ₙ(CH₃)₂(CH₂)ₙN(R³)₂, O(CH₂)ₙN(R³)SOₘA, O(CH₂)ₙN(R³)SOₘN(R³)A, O(CH₂)ₙN(R³)SOₘAr′, (CH₂)ₙN(R³)SOₘA, (CH₂)ₙN(R³)SOₘN(R³)A, (CH₂)ₙN(R³)SOₘAr′, O(CH₂)ₙSOₘA, O(CH₂)ₙSOₘN(R³)A, O(CH₂)ₙSOₘAr′, (CH₂)ₙSOₘA, (CH₂)ₙSOₘN(R³)A, (CH₂)ₙSOₘAr′, —NH—(CH₂)ₙ—NH₂, —NH—(CH₂)ₙ—NHA, —NH—(CH₂)ₙ—NA₂, —NA-(CH₂)ₙ—NH₂, —NA-(CH₂)ₙ—NHA, —NA-(CH₂)ₙ—NA₂, —O—(CH₂)ₙ-Het¹ or Het¹,
R³ denotes H, A or (CH₂)ₙAr′,
R⁴ denotes H, CN, OH, A, (CH₂)ₘAr′, COR³, COAr′, S(O)ₘA or S(O)ₘAr′,
Ar′ denotes phenyl which is unsubstituted or mono-, di-, tri-, tetra- or pentasubstituted by A, Ph, OH, OA, SH, SA, OPh, SPh, NH₂, NHA, NA₂, NHPh, Hal, NO₂, CN, (CH₂)ₙCOOH, (CH₂)ₙCOOA, (CH₂)ₙCONH₂, (CH₂)ₙCONHA, CHO, COA, S(O)ₘA, S(O)ₘPh, NHCOA, NHCOPh, NHSO₂A, NHSO₂Ph or SO₂NH₂,
Ph denotes phenyl which is unsubstituted or mono-, di- or trisubstituted by A, Hal, CN, COOR, COOH, NH₂, NO₂, OH or OA,
Het¹ denotes a monocyclic saturated heterocycle having 1 to 4 N, O and/or S atoms, which may be unsubstituted or mono-, di- or trisubstituted by Hal, A, OA, CN, (CH₂)ₙOH, (CH₂)ₙHal, NH₂, ═NH, ═N—OH, ═N—OA and/or carbonyl oxygen (═O),
A denotes alkyl having 1 to 10 C atoms, where 1-7 H atoms may also be replaced by F and/or chlorine,
Hal denotes F, Cl, Br or I,
n denotes 0, 1, 2, 3, 4 or 5,
m denotes 0, 1 or 2,
   and pharmaceutically usable derivatives, solvates, salts and stereoisomers thereof, including mixtures thereof in all ratios, for the preparation of a medicament for the prophylaxis and/or treatment of diseases in which the inhibition, regulation and/or modulation of kinase signal transduction plays a role.

The compounds of the formula I act as promoters or inhibitors, in particular as inhibitors, of the signalling pathways described herein, preferably as inhibitors of the Raf kinase pathway.

The present invention therefore relates to the use of one or more of the compounds of the formula I for the treatment and/or prophylaxis of diseases, which is characterised in that the diseases are caused, mediated and/or propagated by tyrosine and/or Raf kinase(s).

The compounds of the formula I are particularly effective in diseases which are caused, mediated and/or propagated by the Raf kinases A-Raf, B-Raf and C-Raf-1. The invention therefore furthermore relates to the use of one or more of the compounds of the formula I for the treatment and/or prophylaxis of diseases which are characterised in that they are caused, mediated and/or propagated by A-Raf, B-Raf and/or Raf-1 kinase.

The diseases discussed here are usually divided into two groups, hyperproliferative and non-hyperproliferative diseases.

Hyperproliferative diseases are diseases which are associated with greatly increased cell division, such as, for example, psoriasis, endometriosis, scarring, benign prostate hyperplasia and cancer. Preference is given to the use of one or more of the compounds of the formula I for the prophylaxis and/or treatment of a hyperproliferative disease.

The use of one or more of the compounds of the formula I for the prophylaxis and/or treatment of a hyperproliferative disease which is a cancer-like disease is particularly preferred.

Cancer-like diseases which can be prevented/treated in accordance with the invention using the compounds of the formula I are, in particular, brain cancer, lung cancer, squamous epithelium cancer, bladder cancer, stomach cancer, pancreatic cancer, liver cancer, kidney cancer, colorectal cancer, breast cancer, head cancer, neck cancer, oesophageal cancer, gynaecological cancer, thyroid cancer, lymphoma, chronic leukaemia and acute leukaemia. Particular preference is therefore given to the use of one or more of the compounds of the formula I for the prophylaxis and/or treatment of the cancer-like diseases brain cancer, lung cancer, squamous epithelium cancer, bladder cancer, stomach cancer, pancreatic cancer, liver cancer, kidney cancer, colorectal cancer, breast cancer, head cancer, neck cancer, oesophageal cancer, gynaecological cancer, thyroid cancer, lymphoma, chronic leukaemia and acute leukaemia.

Hyperproliferative diseases which are not cancer-like, but which can be prevented in accordance with the invention using the compounds of the formula I or which can be treated with these compounds are, in particular, psoriasis, endometriosis, scarring, benign prostate hyperplasia. The invention thus furthermore relates to the use of one or more of the compounds of the formula I for the prophylaxis and/or treatment of a hyperproliferative disease which is not cancer-like. The non-cancer-like disease here is preferably psoriasis, endometriosis, scarring or benign prostate hyperplasia.

Diseases which are generally not regarded as hyperproliferative, but against which the compounds of the formula I can be employed include inflammation, arthritis, Helicobacter pylori infection, influenza A, immunological diseases, autoimmune diseases and immunodeficiency disease. The invention therefore also relates to the use of one or more of the compounds of the formula I for the prophylaxis and/or treatment of a disease which is an inflammation, arthritis, a Helicobacter pylori infection, influenza A, an immunological disease, an autoimmune diseases or an immunodeficiency disease.

It can be shown that the compounds of the formula I have an antiproliferative action in vivo in a xenotransplant tumour model. The compounds of the formula I are administered to a patient having a hyperproliferative disease, for example to inhibit tumour growth, to reduce inflammation associated with a lymphoproliferative disease, to inhibit transplant rejection or neurological damage due to tissue repair, etc. The present compounds are suitable for prophylactic or therapeutic purposes. As used herein, the term “treat” is used to refer to both prevention of diseases and treatment of pre-existing conditions. The prevention of proliferation is achieved by administration of the compounds of the formula I prior to the development of overt disease, for example to prevent tumour growth, prevent metastatic growth, diminish restenosis associated with cardiovascular surgery, etc. Alternatively, the compounds are used for the treatment of chronic diseases by stabilising or improving the clinical symptoms of the patient.

The host or patient can belong to any mammalian species, for example a primate species, particularly humans; rodents, including mice, rats and hamsters; rabbits; horses, cows, dogs, cats, etc. Animal models are of interest for experimental investigations, providing a model for treatment of human disease.

The sensitivity of a particular cell to treatment with the compounds of the formula I can be determined by in-vitro testing. Typically, a culture of the cell is combined with a compound according to the invention at various concentrations for a periodine of time which is sufficient to allow the active ingredient to induce cell death or to inhibit migration, usually between about one hour and one week. In-vitro testing can be carried out using cultivated cells from a biopsy sample. The viable cells remaining after the treatment are then counted.

The dose varies depending on the specific compound used, the specific disease, the patient status, etc. A therapeutic dose is typically sufficient considerably to reduce the undesired cell population in the target tissue while the viability of the patient is maintained. The treatment is generally continued until a considerable reduction has occurred, for example an at least about 50% reduction in the cell burden, and may be continued until essentially no more undesired cells are detected in the body.

For the identification of a signal transduction pathway and for detection of interactions between various signal transduction pathways, various scientists have developed suitable models or model systems, for example cell culture models (for example Khwaja et al., EMBO, 1997, 16, 2783-93) and models of transgenic animals (for example White et al., Oncogene, 2001, 20, 7064-7072). For the determination of certain stages in the signal transduction cascade, interacting compounds can be utilised in order to modulate the signal (for example Stephens et al., Biochemical J., 2000, 351, 95-105). The compounds of the formula I can also be used as reagents for testing kinase-dependent signal transduction pathways in animals and/or cell culture models or in the clinical diseases mentioned in this application. Measurement of the kinase activity is a technique which is well known to the person skilled in the art. Generic test systems for the determination of the kinase activity using substrates, for example histone (for example Alessi et al., FEBS Lett. 1996, 399, 3, pages 333-338) or the basic myelin protein are described in the literature (for example Campos-González, R. and Glenney, Jr., J. R. 1992, J. Biol. Chem. 267, page 14535). For the identification of kinase inhibitors, various assay systems are available, for example Walters et al., Nature Drug Discovery 2003, 2; 259-266). In scintillation proximity assay (Sorg et al., J. of. Biomolecular Screening, 2002, 7, 11-19) and flashplate assay, the radioactive phosphorylation of a protein or peptide as substrate with γATP is measured. In the presence of an inhibitory compound, a decreased radioactive signal, or none at all, is detectable. Furthermore, homogeneous time-resolved fluorescence resonance energy transfer (HTR-FRET) and fluoroescence polarisation (FP) technologies are suitable as assay methods (Sills et al., J. of Biomolecular Screening, 2002, 191-214).

Other non-radioactive ELISA assay methods use specific phospho-antibodies (phospho-ABs). The phospho-AB binds only the phosphorylated substrate. This binding can be detected by chemiluminescence using a second peroxidase-conjugated anti-sheep antibody (Ross et al., 2002, Biochem. J., 2002, 977-781).

There are many diseases associated with deregulation of cell proliferation and cell death (apoptosis). The conditions of interest include, but are not limited to, the following. The compounds of the formula I are suitable for the treatment of a variety of conditions where there is proliferation and/or migration of smooth muscle cells and/or inflammatory cells into the intimal layer of a vessel, resulting in restricted blood flow through that vessel, for example in the case of neointimal occlusive lesions. Occlusive graft vascular diseases of interest include atherosclerosis, graft coronary vascular disease after transplantation, vein graft stenosis, peri-anastomotic prosthetic restenosis, restenosis after angioplasty or stent placement, and the like.

Also usable in accordance with the invention are the optically active forms (stereoisomers), the enantiomers, the racemates, the diastereomers and the hydrates and solvates of the compounds of the formula I. The term solvates of the compounds is taken to mean adductions of inert solvent molecules onto the compounds which form owing to their mutual attractive force. Solvates are, for example, mono- or dihydrates or alkoxides.

The term pharmaceutically usable derivatives is taken to mean, for example, the salts of the compounds of the formula I and also so-called prodrug compounds.

The term prodrug derivatives is taken to mean compounds of the formula I which have been modified by means of, for example, alkyl or acyl groups, sugars or oligopeptides and which are rapidly cleaved in the organism to form the effective compounds of the formula I.

These also include biodoegradable polymer derivatives of the compounds of the formula I, as described, for example, in Int. J. Pharm. 115, 61-67 (1995).

The expression “effective amount” denotes the amount of a medicament or of a pharmaceutical active ingredient which causes in a tissue, system, animal or human a biological or medical response which is sought or desired, for example, by a researcher or physician.

In addition, the expression “therapeutically effective amount” denotes an amount which, compared with a corresponding subject who has not received this amount, has the following consequence: improved treatment, healing, prevention or elimination of a disease, syndrome, condition, complaint, disorder or side-effects or also the reduction in the progress of a disease, condition or disorder.

The expression “therapeutically effective amount” also encompasses the amounts which are effective for increasing normal physiological function.

The invention also relates to the use of mixtures of the compounds according to the invention, for example mixtures of two diastereomers, for example in the ratio 1:1, 1:2, 1:3, 1:4, 1:5, 1:10, 1:100 or 1:1000. These are particularly preferably mixtures of stereoisomeric compounds.

Above and below, the radicals Y, Z, Ar¹ and Ar² have the meanings indicated for the formula I, unless expressly stated otherwise.

A denotes alkyl, is unbranched (linear) or branched, and has 1, 2, 3, 4, 5, 6, 7, 8, 9 or 10 C atoms. A preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2- , 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, further preferably, for example, trifluoromethyl.

A very particularly preferably denotes alkyl having 1, 2, 3, 4, 5 or 6 C atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl. A also denotes cycloalkyl.

Cycloalkyl preferably denotes cyclopropyl, cyclobutyl, cylopentyl, cyclohexyl or cycloheptyl.

Alkylene is preferably unbranched and preferably denotes methylene, ethylene, propylene, butylene or pentylene.

R¹ and R², independently of one another, preferably denote, for example, A, such as, for example, methyl or ethyl; Ar′, such as, for example, phenyl, F—, Cl— or bromophenyl or tolyl; OR³, such as, for example, hydroxyl, methoxy or ethoxy; SR³, such as, for example, SCH₃; OAr′, such as, for example, phenoxy; SAr′, such as, for example, S-phenyl; N(R³)₂, such as, for example, amino, methylamino, ethylamino, dimethylamino or diethylamino; NHAr′, such as, for example, anilino; Hal, NO₂, CN, (CH₂)ₙCOOR³, such as, for example, carboxyl, methoxycarbonyl, methoxycarbonylmethyl or ethoxycarbonylethyl; (CH₂)ₙCON(R³)₂, such as, for example, aminocarbonyl, N-methylaminocarbonyl, aminocarbonylmethyl or dimethylaminoethyl; COR³, such as, for example, formyl, acetyl or propionyl; S(O)ₘA, such as, for example, methylsulfonyl; S(O)ₘAr′, such as, for example, phenylsulfonyl; NHCOA, such as, for example, acetamino; NHCOAr′, such as, for example, phenylcarbonylamino; NHSO₂A, such as, for example, methylsulfonylamino; NHSO₂Ar′, such as, for example, phenylsulfonylamino; SOₘN(R³)₂, such as, for example, dimethylaminosulfonyl; —O—(CH₂)ₙ—NH₂, such as, for example, 2-aminoethoxy; —O—(CH₂)ₙ—NHR³, such as, for example, 2-methylaminoethoxy; —O—(CH₂)ₙ—NA₂, such as, for example, 2-dimethylaminoethoxy; O(CH₂)ₙC(CH₃)₂(CH₂)ₙN(R³)₂, such as, for example, OCH₂C(CH₃)₂CH₂NH₂; NH(CH₂)ₙ(CH₃)₂(CH₂)ₙN(R³)₂, such as, for example, NHCH₂(CH₃)₂CH₂NH₂; O(CH₂)ₙN(R³)SOₘA, such as, for example, OCH₂NHSO₂CH₃; O(CH₂)ₙN(R³)SOₘN(R³)A, such as, for example, OCH₂NHSO₂NHCH₃; O(CH₂)ₙN(R³)SOₘAr′, such as, for example, phenylsulfonylaminomethoxy; (CH₂)ₙN(R³)SOₘA, such as, for example, CH₂NHSO₂CH₃; (CH₂)ₙN(R³)SOₘN(R³)A, such as, for example, CH₂NHSO₂NHCH₃; (CH₂)ₙN(R³)SOₘAr′, such as, for example, phenylsulfonylaminomethyl; O(CH₂)ₙSOₘA, such as, for example, O(CH₂)₂SO₂CH₃; O(CH₂)ₙSOₘN(R³)A, such as, for example, OCH₂SO₂NHCH₃; O(CH₂)ₙSOₘAr′, such as, for example, phenylsulfonylmethoxy; (CH₂)ₙSOₘA, such as, for example, CH₂SO₂CH₃; (CH₂)ₙSOₘN(R³)A, such as, for example, CH₂SO₂NHCH₃; (CH₂)ₙSOₘAr′, such as, for example, phenylsulfonylmethyl; —NH—(CH₂)ₙ—NH₂, such as, for example, 2-aminoethylamino; —NH—(CH₂)ₙ—NHA, such as, for example, 2-methylaminoethylamino; —NH—(CH2)ₙ—NA₂, such as, for example, 2-dimethylaminoethylamino; —NA-(CH₂)ₙ—NH₂, such as, for example, (2-aminoethyl)methylamino; —NA-(CH₂)ₙ—NHA, such as, for example, (2-methylaminoethyl)methylamino; —NA-(CH₂)ₙ—NA₂, such as, for example, (2-dimethylaminoethyl)methylamino; —O—(CH₂)ₙ-Het¹, such as, for example, 2-(pyrrolidin-1-yl)ethoxy, 2-(1-piperidin-1-yl)ethoxy, 2-(morpholin-4-yl)ethoxy, 2-(piperazin-1-yl)ethoxy, 2-(4-methylpiperazin-1-yl)ethoxy, 2-(1-methylpiperidin-4-yl)ethoxy, 2-(4-hydroxyethylpiperazin-1-yl)ethoxy or 2-(4-hydroxypiperidin-1-yl)ethoxy; or Het¹, such as, for example, 1-pyrrolidinyl, 1-piperidinyl, 4-morpholinyl, 1-piperazinyl, 4-methylpiperazin-1-yl, 4-piperidinyl, 1-methylpiperidin-4-yl, 4-hydroxyethylpiperazin-1-yl, 4-hydroxypiperidin-1-yl, 2-oxopiperazin-1-yl, 3-oxopiperazin-1-yl, 2-oxomorpholin-4-yl, 3-oxomorpholin-4-yl, 2-pyrrolidon-1-yl, 3-pyrrolidon-1-yl.

R³ preferably denotes H, A or benzyl, particularly preferably with methyl, ethyl, n-propyl, i-propyl, n-butyl, 2-methylpropyl, tert-butyl, and very particularly preferably H.

Ar¹ and Ar², independently of one another, preferably denote unsubstituted phenyl, furthermore phenyl which is mono-, di-, tri-, tetra- or pentasubstituted by A, Ph, OH, OA, SH, SA, OPh, SPh, NH₂, NHA, NA₂, NHPh, Hal, NO₂, CN, (CH₂)ₙCOOH, (CH₂)ₙCOOA, (CH₂)ₙCONH₂, (CH₂)ₙCONHA, (CH₂)ₙCONA₂, CHO, COA, S(O)ₘA, S(O)ₘAr′, NHCOA, NACOAr′, NASO₂A, NASO₂Ph or SO₂NH₂, such as, for example, o-, m- or p-tolyl, biphenyl, o-, m- or p-hydroxyphenyl, o-, m- or p-methoxyphenyl, o-, m- or p-mercaptophenyl, o-, m- or p-phenoxyphenyl, o-, m- or p-anilino, o-, m- or p-methylaminophenyl, o-, m- or p-phenylaminophenyl, o-, m- or p-fluorophenyl, o-, m- or p-chlorophenyl, o-, m- or p-bromophenyl, o-, m- or p-nitrophenyl, o-, m- or p-cyanophenyl, o-, m- or p-carboxyphenyl, o-, m- or p-carboxymethylphenyl, o-, m- or p-methoxycarbonylphenyl, o-, m- or p-methoxycarbonylmethylphenyl, o-, m- or p-aminocarbonylphenyl, o-, m- or p-methylaminocarbonylphenyl, o-, m- or p-formylphenyl, o-, m- or p-acetylphenyl, o-, m- or p-methylsulfonylphenyl, o-, m- or p-methylcarbonylaminophenyl, o-, m- or p-methylsulfonylaminophenyl, o-, m- or p-aminosulfonylphenyl, further preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl or 3-fluoro-4-methoxyphenyl; further, preferably, irrespective of additional substitutions, for example, 2- or 3-furyl, 2- or 3-thienyl, 1-, 2- or 3-pyrrolyl, 1-, 2, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4- or -5-yl or 2,1,3-benzoxadiazol-5-yl.

Ar′ preferably denotes, for example, unsubstituted phenyl, furthermore phenyl which is mono-, di-, tri-, tetra- or pentasubstituted by A, Ph, OH, OA, SH, SA, OPh, SPh, NH₂, NHA, NA₂, NHPh, Hal, NO₂, CN, (CH₂)ₙCOOH, (CH₂)ₙCOOA, (CH₂)ₙCONH₂, (CH₂)ₙCONHA, (CH₂)ₙCONHA₂, CHO, COA, S(O)ₘA, S(O)ₘPh, NACOA, NACOPh, NHSO₂A, NHSO₂Ph or SO₂NH₂, such as, for example, o-, m- or p-tolyl, biphenyl, o-, m- or p-hydroxyphenyl, o-, m- or p-methoxyphenyl, o-, m- or p-mercaptophenyl, o-, m- or p-phenoxyphenyl, o-, m- or p-anilino, o-, m- or p-methylaminophenyl, o-, m- or p-phenylaminophenyl, o-, m- or p-fluorophenyl, o-, m- or p-chlorophenyl, o-, m- or p-bromophenyl, o-, m- or p-nitrophenyl, o-, m- or p-cyanophenyl, o-, m- or p-carboxyphenyl, o-, m- or p-carboxymethylphenyl, o-, m- or p-methoxycarbonylphenyl, o-, m- or p-methoxycarbonylmethylphenyl, o-, m- or p-aminocarbonylphenyl, o-, m- or p-methylaminocarbonylphenyl, o-, m- or p-formylphenyl, o-, m- or p-acetylphenyl, o-, m- or p-methylsulfonylphenyl, o-, m- or p-methylcarbonylaminophenyl, o-, m- or p-methylsulfonylaminophenyl, o-, m- or p-aminosulfonylphenyl, further preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl or 3-fluoro-4-methoxyphenyl.

Het preferably denotes, for example, 2- or 3-furyl, 2- or 3-thienyl, 1-, 2- or 3-pyrrolyl, 1-, 2, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothia-diazol-4- or -5-yl or 2,1,3-benzoxadiazol-5-yl.

In a further preferred embodiment, Het denotes a monocyclic saturated heterocycle having 1 to 3 N, O and/or S atoms, pyridyl is particularly preferred.

Unsubstituted Het¹ preferably denotes, for example, tetrahydro-2- or -3-furyl, 1,3-dioxolan-4-yl, tetrahydro-2- or -3-thienyl, tetrahydro-1-, -2- or -4-imidazolyl, pyrrolidinyl, piperidinyl, morpholinyl or piperazinyl.

Het¹ particularly preferably denotes a monocyclic saturated heterocycle having 1 to 2 N atoms, which may be unsubstituted or monosubstituted by A or (CH₂)ₙOH.

Het¹ very particularly preferably denotes 1-pyrrolidinyl, 1-piperidinyl, 4-morpholinyl, 1-piperazinyl, 4-methylpiperazin-1-yl, 4-piperidinyl, 1-methylpiperidin-4-yl, 4-hydroxyethylpiperazin-1-yl, 4-hydroxypiperidin-1-yl, 2-oxopiperidin-1-yl, 2-oxopyrrolidin-1-yl, 5,5-dimethyl-2-oxopyrrolidin-1-yl, 2-oxopiperazin-1-yl or 3-oxomorpholin-4-yl.

Y particularly preferably denotes O.

Z particularly preferably denotes CH₂, —CHA-O—, —O—, CO, CHEt, CHiPr or CHCH₃.

Hal preferably denotes F, Cl or Br, but also I, particularly preferably F or Cl.

Throughout the invention, all radicals which occur more than once, such as, for example, R¹, R² or R³, may be identical or different, i.e. are independent of one another.

The compounds of the formula I can have one or more chiral centres and can therefore occur in various stereoisomeric forms. The formula I encompasses all these forms.

Accordingly, the formula I encompasses, in particular, the compounds in which at least one of the said radicals has one of the preferred meanings indicated above. Some preferred groups of compounds can be expressed by the following sub-formulae Ia to Ij, which conform to the formula I and in which the radicals not designated in greater detail have the meaning indicated for the formula I, but in which
in Ia Z denotes —CH₂—(CH₂)ₙ—, —(CH₂)ₙ—CHA, —CHA-O— or —O—;
in Ib Ar¹, denotes phenyl which is unsubstituted or mono-, di-, tri-, tetra- or pentasubstituted by R¹
   Ar² denotes Het, phenyl, naphthyl or biphenyl, each of which is unsubstituted or mono-, di-, tri-, tetra- or pentasubstituted by R²;
in Ic R¹, R², independently of one another, denotes A, OH, OA, Hal, S(O)ₘA, NH₂, NHA, NA₂, Hal, (CH₂)ₙCONH₂, (CH₂)ₙCONHA, (CH₂)ₙCONA₂, —O—(CH₂)ₙ—NH₂, —O—(CH₂)ₙ—NHA, —O—(CH₂)ₙ—NA₂, —NH—(CH₂)ₙ—NH₂, —NH—(CH₂)ₙ—NHA, —NH—(CH₂)ₙ—NA₂, —NA-(CH₂)ₙ—NH₂, —NA-(CH₂)ₙ—NHA, —NA-(CH₂)ₙ—NA₂, —O—(CH₂)ₙ-Het¹ or Het¹;
in Id Het denotes a monocyclic aromatic heterocycle having 1 to 3 N, O and/or S atoms;
in Ie Y denotes O;
in If Z denotes —(CH₂)ₙ—, —(CH₂)ₙ—CHA, CHA, —O— or —CHA-O—
   Ar¹, denotes phenyl which is unsubstituted or mono-, di-, tri-, tetra- or pentasubstituted by R¹,
   Ar² denotes Het or phenyl, each of which is unsubstituted or mono-, di-, tri-, tetra- or pentasubstituted by R²,
   R¹, R², independently of one another, denote A, OH, OA, Hal, S(O)ₘA, NH₂, NHA, NA₂, Hal, —O—(CH₂)ₙ—NH₂, —O—(CH₂)ₙ—NHA, —O—(CH₂)ₙ—NA₂, —NH—(CH₂)ₙ—NH₂, —NH—(CH₂)ₙ—NHA, —NH—(CH₂)ₙ—NA₂, —NA-(CH₂)ₙ—NH₂, —NA-(CH₂)ₙ—NHA, —NA-(CH₂)ₙ—NA₂, (CH₂)ₙCONH₂, (CH₂)ₙCONHA, (CH₂)ₙCONA₂, —O—(CH₂)ₙ-Het¹ or Het¹,
   Het denotes a monocyclic aromatic heterocycle having 1 to 3 N, O and/or S atoms,
   Het¹ denotes a monocyclic saturated heterocycle having 1 to 2 N and/or O atoms, which may be unsubstituted or monosubstituted by A or (CH₂)ₙOH,
   Y denotes O,
   A denotes alkyl having 1 to 10 C atoms, where 1-7 H atoms may also be replaced by F and/or chlorine,
   Hal denotes F, Cl, Br or I,
   m denotes 0, 1 or 2,
   n denotes 1, 2, 3, 4 or 5;
in Ig Z denotes —O—, —(CH₂)ₙ—, CHA or —CHA-O—
   Ar¹, denotes phenyl which is unsubstituted or mono-, di-, tri-, tetra- or pentasubstituted by R¹,
   Ar² denotes Het or phenyl, each of which is unsubstituted or mono-, di-, tri-, tetra- or pentasubstituted by R²,
   R¹ denotes A, OH, OA, Hal, or S(O)ₘA,
   R² denotes A, OH, OA, or Hal,
   Het denotes furyl, thienyl, pyrrolyl, imidazolyl, pyrazolyl, oxazolyl, thiazolyl, pyridyl, pyrimidinyl, pyridazinyl or pyrazinyl,
   Y denotes O,
   A denotes alkyl having 1 to 10 C atoms, where 1-7 H atoms may also be replaced by F and/or chlorine,
   Hal denotes F, Cl, Br or I,
   m denotes 0, 1 or 2,
   n denotes 1, 2, or 3;
in Ih Ar¹, denotes phenyl which is unsubstituted or mono-, di-, tri-, tetra- or pentasubstituted by R¹,
   Ar² denotes Het or phenyl, each of which is unsubstituted or mono-, di-, tri-, tetra- or pentasubstituted by R²,
   Z denotes —CH₂—, CHCH₃, —O—, —CHA-O—
   Y denotes O,
   Het denotes a monocyclic aromatic heterocycle having 1 to 3 N, O and/or S atoms,
   R¹ denotes A, OH, OA, Hal, or S(O)ₘA,
   R² denotes A, OH, OA, or Hal,
   A denotes alkyl having 1 to 10 C atoms, where 1-7 H atoms may also be replaced by F and/or chlorine,
   Hal denotes F, Cl, Br or I,
   m denotes 0, 1 or 2;
      and pharmaceutically usable derivatives, salts, solvates, tautomers and stereoisomers thereof, including mixtures thereof in all ratios.

Some of the compounds of the formula I and also the starting materials for their preparation are known and can in addition be prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use may also be made here of variants known per se, which are not mentioned here in greater detail.

If desired, the starting materials can also be formed in situ so that they are not isolated from the reaction mixture, but instead are immediately converted further into the compounds of the formula I.

Compounds of the formula I can preferably be obtained by reacting compounds of the formula II with compounds of the formula III or compounds of the formula IV with compounds of the formula V.

The compounds of the formula I in which Y denotes O, and salts thereof, can be prepared by
a) reacting a compound of the formula II 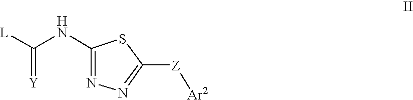
   in which Z and Ar² have the meanings indicated in the formula I, and L denotes Cl, Br, I or a free or reactively functionally modified OH group,
   with a compound of the formula III
      Ar¹—NH₂ III,
   in which Ar¹ has the meaning indicated in the formula I,
      or
b) reacting a compound of the formula IV 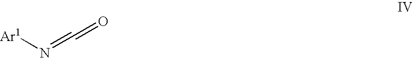
   in which Ar¹ has the meaning indicated in the formula I,
   with a compound of the formula V 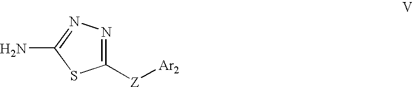
   in which Z and Ar² have the meanings indicated in the formula I,
      and/or
a base or acid of the formula I is converted into one of its salts.

In the compounds of the formula II, L preferably denotes Cl, Br, I or a free or reactively modified OH group, such as, for example, an activated ester, an imidazolide or alkylsulfonyloxy having 1-6 C atoms (preferably methylsulfonyloxy or trifluoromethylsulfonyloxy) or arylsulfonyloxy having 6-10 C atoms (preferably phenyl- or p-tolylsulfonyloxy).

Radicals of this type for activation of the carboxyl group in typical acylation reactions are described in the literature (for example in the standard works, such as Houben-Weyl, Methoden der organischen Chemie [Methods of Organic Chemistry], Georg-Thieme-Verlag, Stuttgart;).

Activated esters are advantageously formed in situ, for example by addition of HOBt or N-hydroxysuccinimide.

Preference is given to the use of compounds of the formula II in which L denotes OH.

The reaction is generally carried out in an inert solvent, in the presence of an acid-binding agent, preferably an organic base, such as DIPEA, triethylamine, dimethylaniline, pyridine or quinoline, or an excess of the carboxyl component of the formula II.

The addition of an alkali or alkaline-earth metal hydroxide, carbonate or bicarbonate or of another salt of a weak acid of the alkali or alkaline-earth metals, preferably of potassium, sodium, calcium or caesium, may also be favourable.

Depending on the conditions used, the reaction time is between a few minutes and 14 days, the reaction temperature is between about 0° and 150°, normally between 15° and 90°, particularly preferably between 15 and 30° C.

Suitable inert solvents are, for example, hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

The compounds of the formula I can be used in their final non-salt form. On the other hand, the present invention also relates to the use of these compounds in the form of their pharmaceutically acceptable salts, which can be derived from various organic and inorganic acids and bases by procedures known in the art. Pharmaceutically acceptable salt forms of the compounds of the formula I are for the most part prepared by conventional methods. If the compound of the formula I contains a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline earth metal hydroxides, such as barium hydroxide and calcium hydroxide; alkali metal alkoxides, for example potassium ethoxide and sodium propoxide; and various organic bases, such as piperidine, diethanolamine and N-methylglutamine. The aluminium salts of the compounds of the formula I are likewise included. In the case of certain compounds of the formula I, acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoarylsulfonates, such as ethanesulfonate, toluenesulfonate and benzenesulfonate, and other organic acids and corresponding salts thereof, such as acetate, trifluoroacetate, tartrate, maleate, succinate, citrate, benzoate, salicylate, ascorbate and the like. Accordingly, pharmaceutically acceptable acid-addition salts of the compounds of the formula I include the following: acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmoate, pectinate, persulfate, phenylacetate, 3-phenylpropionate, phosphate, phosphonate, phthalate, tosylate, but this does not represent a restriction.

Furthermore, the base salts of the compounds of the formula I include aluminium, ammonium, calcium, copper, iron(III), iron(II), lithium, magnesium, manganese(III), manganese(II), potassium, sodium and zinc salts, but this is not intended to represent a restriction. Of the above-mentioned salts, preference is given to ammonium; the alkali metal salts sodium and potassium, and the alkaline earth metal salts calcium and magnesium. Salts of the compounds of the formula I which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris(hydroxymethyl)methylamine (tromethamine), but this is not intended to represent a restriction.

Compounds of the formula I which contain basic nitrogen-containing groups can be quaternised using agents such as (C₁-C₄)alkyl halides, for example methyl, ethyl, isopropyl and tert-butyl chloride, bromide and iodide; di(C₁-C₄)alkyl sulfates, for example dimethyl, diethyl and diamyl sulfate; (C₁₀-C₁₈)alkyl halides, for example decyl, dodecyl, lauryl, myristyl and stearyl chloride, bromide and iodide; and aryl(C₁-C₄)alkyl halides, for example benzyl chloride and phenethyl bromide. Both water- and oil-soluble compounds according to the invention can be prepared using such salts.

The above-mentioned pharmaceutical salts which are preferred include acetate, trifluoroacetate, besylate, citrate, fumarate, gluconate, hemisuccinate, hippurate, hydrochloride, hydrobromide, isethionate, mandelate, meglumine, nitrate, oleate, phosphonate, pivalate, sodium phosphate, stearate, sulfate, sulfosalicylate, tartrate, thiomalate, tosylate and tromethamine, but this is not intended to represent a restriction.

The acid-addition salts of basic compounds of the formula I are prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts otherwise correspond to the respective free base forms thereof.

As mentioned, the pharmaceutically acceptable base-addition salts of the compounds of the formula I are formed with metals or amines, such as alkali metals and alkaline earth metals or organic amines. Preferred metals are sodium, potassium, magnesium and calcium. Preferred organic amines are N,N′-dibenzylethylenediamine, chloroprocaine, choline, diethanolamine, ethylenediamine, N-methyl-D-glucamine and procaine.

The base-addition salts of acidic compounds according to the invention are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts otherwise correspond to the respective free acid forms thereof.

If a compound according to the invention contains more than one group which is capable of forming pharmaceutically acceptable salts of this type, the invention also encompasses multiple salts. Typical multiple salt forms include, for example, bitartrate, diacetate, difumarate, dimeglumine, diphosphate, disodium and trihydrochloride, but this is not intended to represent a restriction.

With regard to that stated above, it can be seen that the term “pharmaceutically acceptable salt” in the present connection is taken to mean an active ingredient which comprises a compound of the formula I in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

Whereas some of the compounds encompassed by the general formula I are known, novel compounds are also included herein. The invention therefore also relates to compounds general formula VI 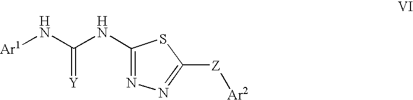
in which
Ar¹ denotes phenyl which is unsubstituted or mono-, di-, tri-, tetra- or pentasubstituted by R¹,
Ar² denotes phenyl or Het, each of which is unsubstituted or mono-, di-, tri-o tetra- or pentasubstituted by R²,
Y denotes O,
Z denotes —O—, —CH₂—(CH₂)ₙ—, —(CH₂)ₙ—CHA-, —CHA-(CH₂)ₙ—, —C(═O)—, —CH(OH)—, —CH(OA)-, —(CH₂)ₙO—, —O(CH₂)ₙ—, —(CH₂)ₙNH— or —NH(CH₂)ₙ—,
Het denotes a mono- or bicyclic aromatic heterocycle having 1 to 4 N, O and/or S atoms,
R¹, R², independently of one another, denote A, OR³, Hal, NO₂, CN, S(O)ₘA, O(CH₂)ₙNA₂ or Het¹,
R³ denotes H or A,
Het¹ denotes a monocyclic saturated heterocycle having 1 to 4 N, O and/or S atoms, which may be unsubstituted or mono-, di- or trisubstituted by Hal, A, OA, CN, (CH₂)ₙOH, (CH₂)ₙHal, NH₂, ═NH, ═N—OH, ═N—OA and/or carbonyl oxygen (═O),
A denotes alkyl having 1 to 10 C atoms, where 1-7 H atoms may also be replaced by F and/or chlorine,
Hal denotes F, Cl, Br or I,
n denotes 0, 1, or 2,
m denotes 0, 1 or 2,
   and pharmaceutically usable derivatives, solvates, salts and stereoisomers thereof, including mixtures thereof in all ratios, which are encompassed by the general formula I.

Particular preference is given to the compounds of the general formula VI in which
Ar¹ denotes phenyl which is unsubstituted or mono-, di-, tri-, tetra- or pentasubstituted by R¹,
Ar² denotes phenyl or Het, each of which is unsubstituted or mono-, di-, tri-, tetra- or pentasubstituted by R²,
Y denotes O,
Z denotes —O—, —CH₂—(CH₂)ₙ—, —(CH₂)ₙ—CHA-, —C(═O)—, —CH(OH)—(CH₂)ₙO—, —O(CH₂)ₙ— or —NH(CH₂)ₙ—,
Het denotes pyridine,
R¹, R², independently of one another, denote A, OR³, Hal, S(O)ₘA, O(CH₂)ₙNA₂ or Het¹,
R³ denotes H or A,
Het¹ denotes pyrimidine,
A denotes alkyl having 1 to 10 C atoms, where 1-7 H atoms may also be replaced by F and/or chlorine,
Hal denotes F, Cl or Br,
n denotes 0, 1, or 2,
m denotes 0, 1 or 2,

The invention furthermore relates to the novel compounds encompassed by the formula I, in particular
1-(2-methoxy-5-trifluoromethylphenyl)-3-(5-pyridin-4-ylmethyl-1,3,4-thiadiazol-2-yl)urea,
1-(5-chloro-2-methoxy-4-methylphenyl)-3-[5-(3,4-dimethoxybenzyl)-1,3,4-thiadiazol-2-yl]urea,
1-[5-(3,4-dimethoxybenzyl)-1,3,4-thiadiazol-2-yl]-3-(3-trifluoromethoxyphenyl)urea,
1-[5-(1-phenylethyl)-1,3,4-thiadiazol-2-yl]-3-(3-trifluoromethanesulfonylphenyl)urea,
1-[5-(3,4-dimethoxybenzyl)-1,3,4-thiadiazol-2-yl]-3-(2-methoxy-5-trifluoromethylphenyl)urea,
1-[5-(1-phenylethyl)-1,3,4-thiadiazol-2-yl]-3-p-tolylurea,
1-(2-methoxy-5-methylphenyl)-3-[5-(1-phenylethyl)-1,3,4-thiadiazol-2-yl]urea,
1-(3-chloro-4-methylphenyl)-3-[5-(1-phenylethyl)-1,3,4-thiadiazol-2-yl]urea,
1-(5-chloro-2-methylphenyl)-3-[5-(1-phenylethyl)-1,3,4-thiadiazol-2-yl]urea,
1-(3-chloro-2-methylphenyl)-3-[5-(1-phenylethyl)-1,3,4-thiadiazol-2-yl]urea,
1-(5-chloro-2-methoxyphenyl)-3-[5-(1-phenylethyl)-1,3,4-thiadiazol-2-yl]urea,
1-[5-(1-phenylethyl)-1,3,4-thiadiazol-2-yl]-3-(3-trifluoromethylphenyl)-urea,
1-[5-(1-phenylethyl)-1,3,4-thiadiazol-2-yl]-3-(4-trifluoromethylphenyl)-urea,
1-[5-(3,4-dimethoxybenzyl)-1,3,4-thiadiazol-2-yl]-3-(2-methoxyphenyl)urea,
1-[5-(1-phenylethyl)-1,3,4-thiadiazol-2-yl]-3-(4-trifluoromethoxyphenyl)urea,
1-(4-fluoro-3-trifluoromethylphenyl)-3-[5-(1-phenylethyl)-1,3,4-thiadiazol-2-yl]urea,
1-(4-chloro-3-trifluoromethylphenyl)-3-[5-(1-phenylethyl)-1,3,4-thiadiazol-2-yl]urea,
1-[5-(2,3-dimethoxybenzyl)-1,3,4-thiadiazol-2-yl]-3-(4-trifluoromethoxyphenyl)urea,
1-[5-(2,3-dimethoxybenzyl)-1,3,4-thiadiazol-2-yl]-3-(2-trifluoromethoxyphenyl)urea,
1-(5-chloro-2,4-dimethoxyphenyl)-3-[5-(3,4-dimethoxybenzyl)-1,3,4-thiadiazol-2-yl]urea,
1-(2,4-dimethoxyphenyl)-3-[5-(1-phenylethyl)-1,3,4-thiadiazol-2-yl]urea,
1-(3-chloro-4-methoxyphenyl)-3-[5-(1-phenylethyl)-1,3,4-thiadiazol-2-yl]urea,
1-[2-(2-dimethylaminoethoxy)-5-trifluoromethylphenyl]-3-[5-(1-phenyl-ethyl)-1,3,4-thiadiazol-2-yl]urea,
1-[4-chloro-5-methyl-2-(piperidin-4-yloxy)phenyl]-3-[5-(3,4-dimethoxy-benzyl)-1,3,4-thiadiazol-2-yl]urea 3d,
1-(2-methoxy-5-trifluoromethylphenyl)-3-[5-(1-phenylethyl)-1,3,4-thiadiazol-2-yl]urea 57,
1-(5-chloro-2-methoxy-4-methylphenyl)-3-(5-pyridin-4-ylmethyl-1,3,4-thiadiazol-2-yl)urea 58,
1-(5-pyridin-4-ylmethyl-1,3,4-thiadiazol-2-yl)-3-(3-trifluoromethoxyphenyl)urea 59,
1-(5-chloro-2-methoxy-4-methylphenyl)-3-[5-(1-phenylethyl)-1,3,4-thiadiazol-2-yl]urea 60,
1-[5-(1-phenylethyl)-1,3,4-thiadiazol-2-yl]-3-(3-trifluoromethoxyphenyl)urea 61,
1-(2-methoxy-5-trifluoromethylphenyl)-3-[5-(1-phenylpropyl)-1,3,4-thiadiazol-2-yl]urea 62,
1-(5-chloro-2-methoxy-4-methylphenyl)-3-[5-(4-chlorophenoxymethyl)-1,3,4-thiadiazol-2-yl]urea 63,
1-[5-(4-chlorophenoxymethyl)-1,3,4-thiadiazol-2-yl]-3-(3-trifluoromethoxyphenyl)urea 64,
1-[4-chloro-2-(2-dimethylaminoethoxy)-5-methylphenyl]-3-[5-(1-phenylethyl)-1,3,4-thiadiazol-2-yl]urea 65,
1-[4-chloro-2-(2-dimethylaminoethoxy)-5-methylphenyl]-3-[5-(3,4-dimethoxybenzyl)-1,3,4-thiadiazol-2-yl]urea 66,
1-[5-(3,4-dimethoxybenzyl)-1,3,4-thiadiazol-2-yl]-3-[2-(2-dimethylaminoethoxy)-5-trifluoromethylphenyl]urea 67,
1-(2-methoxy-5-methylphenyl)-3-[5-(1-phenylpropyl)-1,3,4-thiadiazol-2-yl]urea 68,
1-(2,5-dimethoxyphenyl)-3-[5-(1-phenylethyl)-1,3,4-thiadiazol-2-yl]urea 70,
1-(2,5-dichlorophenyl)-3-[5-(1-phenylethyl)-1,3,4-thiadiazol-2-yl]urea 71,
1-[5-(hydroxyphenylmethyl)-1,3,4-thiadiazol-2-yl]-3-(3-trifluoromethylphenyl)urea 72,
1-(2-methoxy-5-methylphenyl)-3-[5-(2-methyl-1-phenylpropyl)-1,3,4-thiadiazol-2-yl]urea 73,
1-(2-fluoro-5-trifluoromethylphenyl)-3-(5-pyridin-4-ylmethyl-1,3,4-thiadiazol-2-yl)urea 74,
1-(4-fluoro-3-trifluoromethylphenyl)-3-(5-pyridin-4-ylmethyl-1,3,4-thiadiazol-2-yl)urea 75,
1-[5-(3,4-dimethoxybenzoyl)-1,3,4-thiadiazol-2-yl]-3-m-tolylurea 76,
1-{5-[2-(3,4-dimethoxyphenyl)ethyl]-1,3,4-thiadiazol-2-yl}-3-m-tolylurea 77,
1-(3-chloro-4-methylphenyl)-3-[5-(2-methyl-1-phenylpropyl)-1,3,4-thiadiazol-2-yl]urea 78,
1-(3-chlorophenyl)-3-[5-(3,4-dimethoxyphenoxy)-1,3,4-thiadiazol-2-yl]urea 79,
1-(3-chlorophenyl)-3-[5-(3,4-dimethoxybenzoyl)-1,3,4-thiadiazol-2-yl]urea 80,
1-(3-chlorophenyl)-3-{5-[2-(3,4-dimethoxyphenyl)ethyl]-1,3,4-thiadiazol-2-yl}urea 81,
1-(5-chloro-2,4-dimethoxyphenyl)-3-[5-(1-phenylethyl)-1,3,4-thiadiazol-2-yl]urea 82,
1-(3-chlorophenyl)-3-[5-(3,4-dimethoxybenzylamino)-1,3,4-thiadiazol-2-yl]urea 83,
1-[5-(3,4-dimethoxyphenylamino)-1,3,4-thiadiazol-2-yl]-3-(3-trifluoromethylphenyl)urea 84,
1-[5-(3,4-dimethoxyphenoxy)-1,3,4-thiadiazol-2-yl]-3-(3-trifluoromethylphenyl)urea 85,
1-[5-(4-chlorophenoxymethyl)-1,3,4-thiadiazol-2-yl]-3-(4-fluoro-3-trifluoromethylphenyl)urea 86,
1-(5-chloro-2-methoxyphenyl)-3-{5-[2-(3,4-dimethoxyphenyl)ethyl]-1,3,4-thiadiazol-2-yl}urea 87,
1-(5-chloro-2-methoxyphenyl)-3-[5-(3,4-dimethoxybenzoyl)-1,3,4-thiadiazol-2-yl]urea 88,
1-(5-chloro-2-methoxyphenyl)-3-[5-(3,4-dimethoxybenzylamino)-1,3,4-thiadiazol-2-yl]urea 89,
1-{5-[2-(3,4-dimethoxyphenyl)ethyl]-1,3,4-thiadiazol-2-yl}-3-(3-trifluoromethylphenyl)urea 90,
1-[5-(3,4-dimethoxybenzylamino)-1,3,4-thiadiazol-2-yl]-3-(3-trifluoromethylphenyl)urea 91,
1-[5-(3,4-dimethoxyphenylamino)-1,3,4-thiadiazol-2-yl]-3-(2-fluoro-3-trifluoromethylphenyl)urea 92,
1-[5-(3,4-dimethoxyphenoxy)-1,3,4-thiadiazol-2-yl]-3-(2-fluoro-3-trifluoromethylphenyl)urea 93,
1-[5-(3,4-dimethoxyphenoxy)-1,3,4-thiadiazol-2-yl]-3-(4-fluoro-3-trifluoromethylphenyl)urea 94,
1-[5-(3,4-dimethoxybenzoyl)-1,3,4-thiadiazol-2-yl]-3-(3-fluoro-5-trifluoromethylphenyl)urea 95,
1-{5-[2-(3,4-dimethoxyphenyl)ethyl]-1,3,4-thiadiazol-2-yl}-3-(3-fluoro-5-trifluoromethylphenyl)urea 96,
1-[5-(3,4-dimethoxybenzoyl)-1,3,4-thiadiazol-2-yl]-3-(2-fluoro-5-trifluoromethylphenyl)urea 97,
1-[5-(3,4-dimethoxybenzoyl)-1,3,4-thiadiazol-2-yl]-3-(4-fluoro-3-trifluoromethylphenyl)urea 98,
1-[5-(3,4-dimethoxybenzoyl)-1,3,4-thiadiazol-2-yl]-3-(2-fluoro-3-trifluoromethylphenyl)urea 99,
1-{5-[2-(3,4-dimethoxyphenyl)ethyl]-1,3,4-thiadiazol-2-yl}-3-(4-fluoro-3-trifluoromethylphenyl)urea 100,
1-{5-[2-(3,4-dimethoxyphenyl)ethyl]-1,3,4-thiadiazol-2-yl}-3-(2-fluoro-3-trifluoromethylphenyl)urea 101,
1-{5-[2-(3,4-dimethoxyphenyl)ethyl]-1,3,4-thiadiazol-2-yl}-3-(2-fluoro-5-trifluoromethylphenyl)urea 102,
1-[5-(3,4-dimethoxybenzylamino)-1,3,4-thiadiazol-2-yl]-3-(2-fluoro-3-trifluoromethylphenyl)urea 103,
1-(4-chloro-3-trifluoromethylphenyl)-3-{5-[2-(3,4-dimethoxyphenyl)ethyl]-1,3,4-thiadiazol-2-yl}urea 104,
1-(4-chloro-3-trifluoromethylphenyl)-3-[5-(3,4-dimethoxybenzoyl)-1,3,4-thiadiazol-2-yl]urea 105,
1-(4-chloro-3-trifluoromethylphenyl)-3-[5-(3,4-dimethoxybenzylamino)-1,3,4-thiadiazol-2-yl]urea 106,
1-(3,5-bistrifluoromethylphenyl)-3-[5-(3,4-dimethoxyphenylamino)-1,3,4-thiadiazol-2-yl]urea 107,
1-(3,5-bistrifluoromethylphenyl)-3-[5-(3,4-dimethoxybenzoyl)-1,3,4-thiadiazol-2-yl]urea 108,
1-(3,5-bistrifluoromethylphenyl)-3-{5-[2-(3,4-dimethoxyphenyl)ethyl]-1,3,4-thiadiazol-2-yl}urea 109,
1-(3,5-bistrifluoromethylphenyl)-3-[5-(3,4-dimethoxybenzylamino)-1,3,4-thiadiazol-2-yl]urea 110,
1-(3-chlorophenyl)-3-[5-(pyridin-4-yloxy)-1,3,4-thiadiazol-2-yl]urea 111,
1-[5-(pyridin-4-yloxy)-1,3,4-thiadiazol-2-yl]-3-(3-trifluoromethylphenyl)-urea 112,
1-(4-fluoro-3-trifluoromethylphenyl)-3-[5-(pyridin-4-yloxy)-1,3,4-thiadiazol-2-yl]urea 113,
1-(2-fluoro-3-trifluoromethylphenyl)-3-[5-(pyridin-4-yloxy)-1,3,4-thiadiazol-2-yl]urea 114,
1-(2-fluoro-5-trifluoromethylphenyl)-3-[5-(pyridin-4-yloxy)-1,3,4-thiadiazol-2-yl]urea 115,
1-(3,5-bistrifluoromethylphenyl)-3-[5-(pyridin-4-yloxy)-1,3,4-thiadiazol-2-yl]urea 116
1-(5-chloro-2-methoxyphenyl)-3-[5-(4-chlorophenoxymethyl)-1,3,4-thiadiazol-2-yl]urea 117,
1-[5-(4-chlorophenoxymethyl)-1,3,4-thiadiazol-2-yl]-3-(3-trifluoromethylphenyl)urea 118,
1-[5-(3,4-dimethoxybenzoyl)-1,3,4-thiadiazol-2-yl]-3-(3-trifluoromethylphenyl)-urea 119,
1-[5-(3,4-dimethoxyphenoxymethyl)-1,3,4-thiadiazol-2-yl]-3-m-tolylurea 120,
1-(3-chlorophenyl)-3-[5-(3,4-dimethoxyphenoxymethyl)-1,3,4-thiadiazol-2-yl]urea 121,
1-(5-chloro-2-methoxyphenyl)-3-[5-(3,4-dimethoxyphenoxymethyl)-1,3,4-thiadiazol-2-yl]urea 122,
1-[5-(3,4-dimethoxyphenoxymethyl)-1,3,4-thiadiazol-2-yl]-3-(3-trifluoromethylphenyl)urea 123,
1-[5-(3,4-dimethoxyphenoxymethyl)-1,3,4-thiadiazol-2-yl]-3-(2-fluoro-3-trifluoromethylphenyl)urea 124,
1-[5-(3,4-dimethoxyphenoxymethyl)-1,3,4-thiadiazol-2-yl]-3-(3-fluoro-5-trifluoromethylphenyl)urea 125,
1-[5-(3,4-dimethoxyphenoxymethyl)-1,3,4-thiadiazol-2-yl]-3-(4-fluoro-3-trifluoromethylphenyl)urea 126,
1-[5-(3,4-dimethoxyphenoxymethyl)-1,3,4-thiadiazol-2-yl]-3-(2-fluoro-5-trifluoromethylphenyl)urea 127,
1-(4-chloro-3-trifluoromethylphenyl)-3-[5-(3,4-dimethoxyphenoxymethyl)-1,3,4-thiadiazol-2-yl]urea 128,
1-(3,5-bistrifluoromethylphenyl)-3-[5-(3,4-dimethoxyphenoxymethyl)-1,3,4-thiadiazol-2-yl]urea 129,
(S)-1-[5-(1-phenylethyl)-1,3,4-thiadiazol-2-yl]-3-(3-trifluoromethylphenyl)urea 130,
(R)-1-[5-(1-phenylethyl)-1,3,4-thiadiazol-2-yl]-3-(3-trifluoromethylphenyl)urea 131,
(S)-1-(5-chloro-2-methoxyphenyl)-3-[5-(1-phenylethyl)-1,3,4-thiadiazol-2-yl]urea enantiomer 132,
(R)-1-(5-chloro-2-methoxyphenyl)-3-[5-(1-phenylethyl)-1,3,4-thiadiazol-2-yl]urea 133,
(S)-1-(4-fluoro-3-trifluoromethylphenyl)-3-[5-(1-phenylethyl)-1,3,4-thiadiazol-2-yl]urea 134,
(R)-1-(4-fluoro-3-trifluoromethylphenyl)-3-[5-(1-phenylethyl)-1,3,4-thiadiazol-2-yl]urea 135.

The invention also relates to a process for the preparation of the above-mentioned novel compounds and pharmaceutically usable derivatives, salts, solvates and stereoisomers thereof, which is characterised in that
a) a compound of the formula II 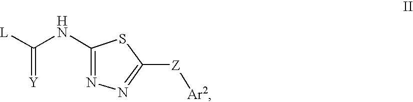
   in which Y, Z and Ar² each have the same meaning as in the respective compound to be prepared,
   and L denotes Cl, Br, I or a free or reactively functionally modified OH group,
   is reacted with a compound of the formula III
      Ar¹—NH₂ III,
   in which Ar¹ has the same meaning as in the respective compound to be prepared,
      or
b) a compound of the formula IV 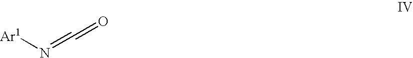
   in which Ar¹ has the same meaning as in the respective compound to be prepared,
   is reacted with a compound of the formula V 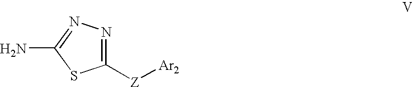
   in which Z and Ar² each have the same meaning as in the respective compound to be prepared,
      and/or
a base or acid of the formula I is converted into one of its salts.

The invention furthermore relates to a medicament comprising at least one of the above-mentioned novel compounds and/or pharmaceutically usable derivatives, solvates and stereoisomers thereof, including mixtures thereof in all ratios, and optionally excipients and/or adjuvants.

Pharmaceutical formulations can be administered in the form of dosage units which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The compounds of the formula I can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compound. Syrups can be prepared by dissolving the compound in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like. The compounds of the formula I and salts, solvates and physiologically functional derivatives thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds of the formula I and the salts, solvates and physiologically functional derivatives thereof can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds may furthermore be coupled to a class of biodoegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye indude eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurised dispensers with aerosols, nebulisers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary.

Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound of the formula I depends on a number of factors, including, for example, the age and weight of the human or animal, the precise condition which requires treatment, and its severity, the nature of the formulation and the method of administration, and is ultimately determined by the treating doctor or vet. However, an effective amount of a compound according to the invention for the treatment of neoplastic growth, for example colon or breast carcinoma, is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as a single dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound according to the invention per se. It can be assumed that similar doses are suitable for the treatment of other conditions mentioned above.

The invention also relates to a set (kit) consisting of separate packs of
(a) an effective amount of a compound of the formula I and/or pharmaceutically usable derivatives, solvates and stereoisomers thereof, including mixtures thereof in all ratios,
and
(b) an effective amount of a further medicament active ingredient.

The set comprises suitable containers, such as boxes, individual bottles, bags or ampoules. The set may, for example, comprise separate ampoules, each containing an effective amount of a compound of the formula I and/or pharmaceutically usable derivatives, solvates and stereoisomers thereof, including mixtures thereof in all ratios, and an effective amount of a further medicament active ingredient in dissolved or lyophilised form.

The compounds of the formula I are also suitable for combination with known anti-cancer agents. These known anti-cancer agents include the following: oestrogen receptor modulators, androgen receptor modulators, retinoid receptor modulators, cytotoxic agents, antiproliferative agents, prenyl-protein transferase inhibitors, HMG-COA reductase inhibitors, HIV protease inhibitors, reverse transcriptase inhibitors and further angiogenesis inhibitors. The present compounds are particularly suitable for administration at the same time as radiotherapy.

“Oestrogen receptor modulators” refers to compounds which interfere with or inhibit the binding of oestrogen to the receptor, regardless of mechanism. Examples of oestrogen receptor modulators include, but are not limited to, tamoxifen, raloxifene, idoxifene, LY353381, LY 117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]phenyl 2,2-dimethylpropanoate, 4,4′-dihydroxybenzophenone-2,4-dinitrophenylhydrazone and SH646. “Androgen receptor modulators” refers to compounds which interfere with or inhibit the binding of androgens to the receptor, regardless of mechanism. Examples of androgen receptor modulators include finasteride and other 5α-reductase inhibitors, nilutamide, flutamide, bicalutamide, liarozole and abiraterone acetate.

“Retinoid receptor modulators” refers to compounds which interfere with or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl)retinamide and N-4-carboxyphenyl retinamide.

“Cytotoxic agents” refers to compounds which result in cell death primarily through direct action on the cellular function or which inhibit or interfere with cell myosis, including alkylating agents, tumour necrosis factors, intercalators, microtubulin inhibitors and topoisomerase inhibitors. Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosylate, trofosfamide, nimustine, dibrospidium chloride, pumitepa, lobaplatin, satraplatin, profiromycin, cisplatin, irofulven, dexifosfamide, cis-aminedichloro(2-methylpyridine)platinum, benzylguanine, glufosfamide, GPX100, (trans,trans,trans)bis-mu-(hexane-1,6-diamine)mu-[diamine-platinum(II)]bis[diamine(chloro)platinum(II)]tetrachloride, diarisidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-de-amino-3′-morpholino-13-deoxo-10-hydroxycarminomycin, annamycin, galarubicin, elinafide, MEN10755 and 4-demethoxy-3-deamino-3-aziridinyl-4-methylsulfonyldaunorubicin (see WO 00/50032).

Examples of microtubulin inhibitors include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzenesulfonamide, anhydrovinblastine, N,N-dimethyl-L-valyl-L-valyl-N-methyl-L-valyl-L-prolyl-L-proline-t-butylamide, TDX258 and BMS188797.

Some examples of topoisomerase inhibitors are topotecan, hycaptamine, irinotecan, rubitecan, 6-ethoxypropionyl-3′,4′-O-exobenzylidenechartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H,12H-benzo[de]pyrano[3′,4′:b,7]indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxyetoposide, GL331, N-[2-(dimethylamino)-ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]-acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one and dimesna.

“Antiproliferative agents” include antisense RNA and DNA oligonucleotides such as G3139, ODN698, RVASKRAS, GEM231 and INX3001 and anti-metabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, neizarabine, 2′-deoxy-2′-methylidenecytidine, 2′-fluoromethylene-2′-deoxycytidine, N-[5-(2,3-di-hydrobenzofuryl)sulfonyl]-N′-(3,4-dichlorophenyl)urea, N6-[4-deoxy-4-[N2-[2(E),4(E)-tetradecadienoyl]glycylamino]-L-glycero-B-L-mannohepto-pyranosyl]adenine, aplidine, ecteinascidin, troxacitabine, 4-[2-amino-4-oxo-4,6,7,8-tetrahydro-3H-pyrimidino[5,4-b]-1,4-thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo-(7.4.1.0.0)tetradeca-2,4,6-trien-9-ylacetic acid ester, swainsonine, lometrexol, dexrazoxane, methioninase, 2′-cyano-2′-deoxy-N4-palmitoyl-1-B-D-arabinofuranosyl cytosine and 3-aminopyridine-2-carboxaldehyde thiosemicarbazone. “Antiproliferative agents” also include monoclonal antibodies to growth factors other than those already listed under “angiogenesis inhibitors”, such as trastuzumab, and tumour suppressor genes, such as p53, which can be delivered via recombinant virus-mediated gene transfer (see U.S. Pat. No. 6,069,134, for example).

The assays are known from the literature and can easily be carried out by the person skilled in the art (see, for example, Dhanabal et al., Cancer Res. 59:189-197; Xin et al., J. Biol. Chem. 274:9116-9121; Sheu et al., Anticancer Res. 18:4435-4441; Ausprunk et al., Dev. Biol. 38:237-248; Gimbrone et al., J. Natl. Cancer Inst. 52:413-427; Nicosia et al., In Vitro 18:538-549).

Above and below, all temperatures are indicated in ° C. In the following examples, “conventional work-up” means: water is added if necessary, the pH is adjusted, if necessary, to a value of between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. Rf values on silica gel; eluent: ethyl acetate/methanol 9:1.

Mass spectrometry (MS): EI (electron impact ionisation) M⁺ FAB (fast atom bombardment) (M+H)⁺ ESI (electrospray ionisation) (M+H)⁺ APCI-MS (atmospheric pressure chemical ionization—mass spectrometry) (M+H)⁺.

### I) Synthesis of Thiadiazole Units 1a-h

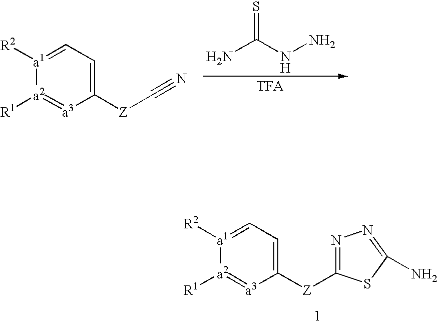

34 mmol of nitrile and 3.3 eq of thiosemicarbazide is dissolved in 9 eq of trifluoroacetic acid and stirred overnight. The reaction mixture is subsequently added to water and neutralised using 32% ammonia solution. The deposited precipitate is filtered off with suction and washed with water. The precipitate is dried overnight at 50° C. and 100 mbar. Substituents and yields:
1a: R¹=R²=OMe, Z=CH₂, a¹=a²=a³=C; 7.2 g (70%) of colourless solid; LC-MS (m/e): 252.2, HPLC: 2.58 min
1b: R¹⁼R²⁼H, Z=CHCH₃, a¹=a²=a³=C; 2.7 g (35%) of colourless solid; LC-MS (m/e): 206.2, HPLC: 2.64 min
1c: R¹⁼R²⁼H, Z=CH₂, a¹=N, a²=a³=C; 2.1 g (49%) of colourless solid; LC-MS (m/e): 193.2, HPLC: 0.63 min
1d: R¹⁼R²⁼H, Z=CH₂, a¹=a²=C, a³=N; 0.3 g (20%) of colourless solid; LC-MS (m/e): 193.2, HPLC: 0.47 min
1e: R¹⁼H, R²⁼Cl, Z=—O—CH₂—, a¹=a²=a³=C; 2.8 g (89%) of colourless solid
1f: R¹⁼R²⁼H, Z=—CH(OH)—, a¹=a²=a³=C; 0.7 g (7%) of colourless solid
1g: R¹⁼R²⁼H, Z=—CH(Et)-, a¹=a²=a³=C; 0.5 g (33%) of colourless solid
1h: R¹⁼R²⁼H, Z=—CH(iPr)-, a¹=a²=a³=C; 0.5 g (6%) of colourless solid

### Synthesis of Thiadiazole Unit 1i

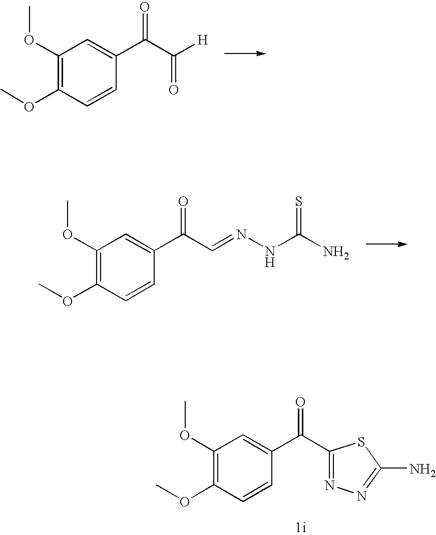

Thiosemicarbazide (0.91 g, 10 mmol) is added at 0° C. to a solution of 3,4-dimethoxyphenylglyoxal (1.94 g, 10 mmol) in water (150 ml). After minutes, the orange precipitate is filtered and used further in the next step without further purification (1.3 g, 49%).

Iron(III) chloride (6 g, 22 mmol) in water (50 ml) is added to a suspension of 4-(3,4-dimethoxyphenyl)thiosemicarbazone (1.3 g, 8.6 mmol) in water (50 ml). The mixture is refluxed for one hour. After cooling, the brown precipitate is filtered and dried in vacuo, giving (5-amino-1,3,4-thiadiazol-2-yl)-(3,4-dimethoxyphenyl)methanone Ii as ochre-coloured powder (1.7 g, 74%).

### Synthesis of Thiadiazole Unit 1j

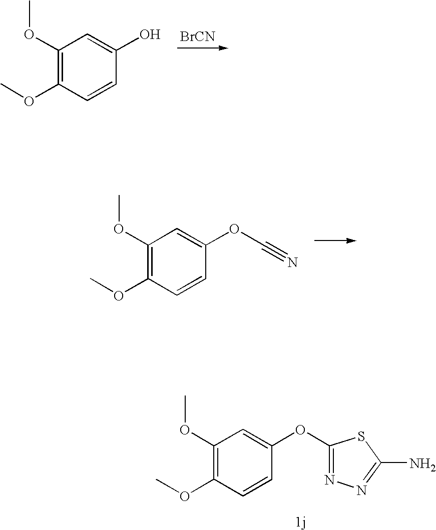

Triethylamine (3 ml, 20 mmol) is added to a solution of 3,4-dimethoxyphenol (3.08 g, 20 mmol) in diethyl ether (40 ml). The reaction solution is cooled to −5° C., and a solution of cyanogen bromide (2.32 g, mmol) in diethyl ether (20 mmol) is added dropwise. The reaction solution is stirred at −5° C. for one hour. The resultant precipitate is filtered off, and the filtrate is evaporated in vacuo. The precipitate is triturated with diethyl ether and filtered. The precipitate is dried in vacuo, giving 4-cyanato-1,2-dimethoxybenzene (1.8 g, 50%) as colourless needles.

Thiosemicarbazide (0.92 g, 10 mmol) is added to a solution of 4-cyanato-1,2-dimethoxybenzene (1.80 g, 10 mmol) in trifluoroacetic acid (40 ml), and the reaction solution is refluxed for six hours. After cooling, the mixture is neutralised using 10% ammonia. The reaction solution is extracted with ethyl acetate, and the organic phase is then extracted with water. The organic phase is dried over magnesium sulfate, and the solvent is removed in vacuo. The precipitate is triturated with diethyl ether and filtered, giving 5-(3,4-dimethoxyphenoxy)-1,3,4-thiadiazol-2-ylamine 1j (0.15 g, 6%) as grey powder.

### Synthesis of Thiadiazole Unit 1k

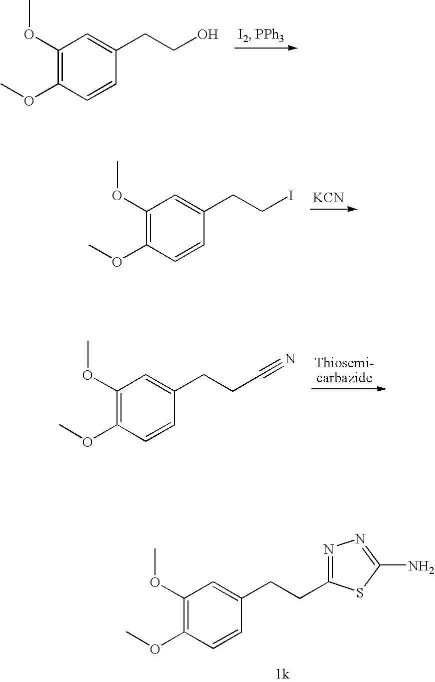

3,4-Dimethoxyphenethyl alcohol (1.82 g, 10 mmol), triphenylphosphine (3.14 g, 12 mmol), imidazole (0.82 g, 12 mmol) and iodine (2.9 g, 11.5 mmol) are dissolved in anhydrous toluene (50 ml) and stirred for 24 h at room temperature under nitrogen. The reaction mixture is subsequently hydrolysed using sodium thiosulfate. The organic phase is washed with saturated potassium carbonate solution and dried over magnesium sulfate, and the solvent is removed in vacuo. The residue is purified by means of column chromatography (ethyl acetate/cyclohexane 1:4) to give 4-(2-iodoethyl)-1,2-dimethoxybenzene (2.9 g, 100%) as colourless oil.

Potassium cyanide (650 mg, 10 mmol) is added to a solution of 4-(2-iodoethyl)-1,2-dimethoxybenzene (2.92 g, 10 mmol) in ethanol/water (75 ml/7.5 ml). The reaction solution is refluxed overnight, and the solvent is subsequently removed in vacuo. The residue is taken up in water and extracted with diethyl ether. The combined organic phases are washed with water, dried over magnesium sulfate, and the solvent is removed in vacuo, giving 3-(3,4-dimethoxyphenyl)propionitrile (1.9 g, 97%) as colourless oil.

Thiosemicarbazide (0.92 g, 10 mmol) is added to a solution of 3-(3,4-dimethoxyphenyl)propionitrile (1.91 g, 10 mmol) in trifluoroacetic acid (40 ml), and the reaction solution is refluxed for six hours. After cooling, the mixture is neutralised using 10% ammonia. The precipitate is filtered off and washed firstly with diethyl ether and then with ethyl acetate, giving 5-[2-(3,4-dimethoxyphenyl)ethyl]-1,3,4-thiadiazol-2-ylamine 1k (1.37 g, 52%) as white needles.

### Synthesis of Thiadiazole Unit 1l

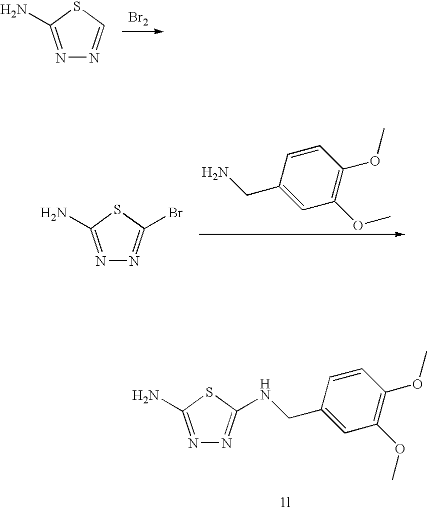

Aminothiadiazole (2.1 g, 20 mmol) is dissolved in glacial acetic acid (10 ml). Bromine (3.65 g, 1.2 ml, 22 mmol) is subsequently added over the course of 30 min, and the reaction solution is stirred at room temperature for 18 hours. The solvent is removed in vacuo, and the residue is taken up with water, rendered basic using sodium hydrogencarbonate and extracted with ethyl acetate. The combined organic phases are washed with aqueous sodium thiosulfate solution, dried over magnesium sulfate, and the solvent is removed in vacuo, giving 5-bromo-1,3,4-thiadiazol-2-ylamine (2.43 g, 68%) as yellow powder, which is used further in the next step without further purification. A mixture of veratrylamine (1.0 g, 5.98 mmol), 5-bromo-1,3,4-thiadiazol-2-ylamine (1.08 g, 5.98 mmol) and potassium carbonate (1.0 g, 5.98 mmol) is dissolved in ethanol (100 ml) and stirred at room temperature for 24 hours. After the solvent has been removed in vacuo, the residue is taken up with water and extracted with ethyl acetate. The organic phase is subsequently washed with saturated sodium chloride solution, dried over magnesium sulfate, and the solvent is removed in vacuo, giving N-(3,4-dimethoxybenzyl)-1,3,4-thiadiazole-2,5-diamine 1l (1.48 g, 93%) as colourless solid. It is employed in the subsequent steps without further purification.

### Synthesis of Thiadiazole Unit 1m

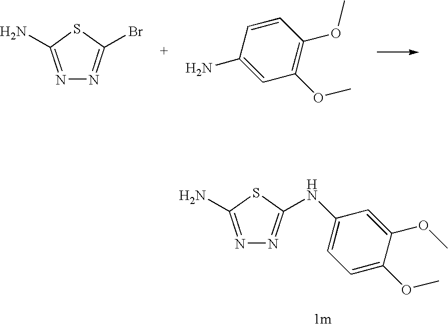

A mixture of aminoveratrol (1.53 g, 10.0 mmol), 5-bromo-1,3,4-thiadiazol-2-ylamine (1.8 g, 10.0 mmol) and potassium carbonate (1.38 g, 10.0 mmol) is dissolved in ethanol (50 ml) and stirred at room temperature for 18 hours. After removal of the solvent in vacuo, the residue is taken up with water and extracted with ethyl acetate. The organic phase is subsequently washed with saturated sodium chloride solution, dried over magnesium sulfate, and the solvent is removed in vacuo. The residue is purified by means of column chromatography (ethyl acetate/methanol/triethylamine 9:0.9:0.1) to give N-(3,4-dimethoxyphenyl)-1,3,4-thiadiazole-2,5-diamine 1m (2.5 g, 99%) as pale-pink needles.

### Synthesis of Thiadiazole Unit 1n

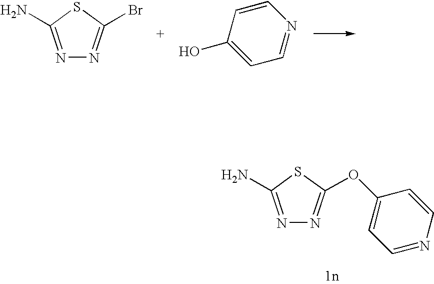

After removal of the solvent in vacuo, the residue is taken up with water and extracted with ethyl acetate. The organic phase is subsequently washed with saturated sodium chloride solution, dried over magnesium sulfate, and the solvent is removed in vacuo.

t-BuOK (840 mg, 7.50 mmol) is added to hydroxypyridine (475 mg, 5.0 mmol) in dry dimethylformamide (10 ml). After stirring at room temperature for two hours, 5-bromo-1,3,4-thiadiazol-2-ylamine (900 mg, 5.0 mmol) is added, and the reaction solution is heated at 80° C. for 12 hours. The solvent is subsequently removed in vacuo, the residue is taken up with water and filtered. The product is dried in vacuo, giving N-(3,4-dimethoxyphenyl)-1,3,4-thiadiazole-2,5-diamine (290 mg, 30%) as grey powder.

### Synthesis of of Thiadiazole Unit 1o

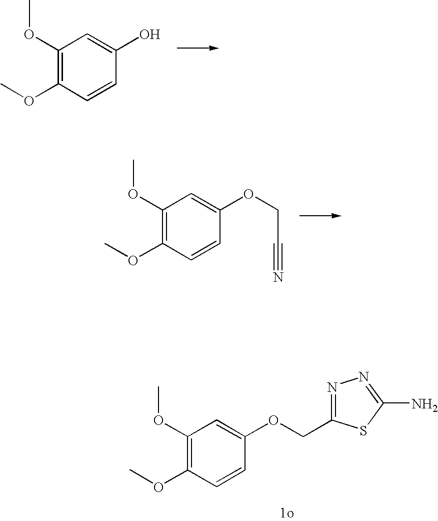

Potassium carbonate (5.6 g, 40 mmol) is added to a solution of 3,4-dimethoxyphenol (3.08 g, 20 mmol) in acetone (40 ml). A solution of bromoacetonitrile (1.40 ml, 20 mmol) in acetone (10 ml) is subsequently added dropwise and refluxed for five hours. The precipitate is filtered off, and the solvent of the filtrate is removed in vacuo. Purification by means of column chromatography (ethyl acetate/cyclohexane 1:1) gives 3,4-dimethoxyphenoxyacetonitrile (3.77 g, 98%) as white needles.

Thiosemicarbazide (2.0 g, 22 mmol) is added to a solution of 3,4-dimethoxyphenoxyacetonitrile (3.86 g, 20 mmol) in trifluoroacetic acid (25 ml), and the reaction solution is refluxed for six hours. After cooling, the reaction solution is neutralised using 10% ammonia, and the precipitate is filtered off. Washing of the precipitate with acetone and diethyl ether gives 5-(3,4-dimethoxyphenoxymethyl)-1,3,4-thiadiazol-2-ylamine (4.60 g, 86%) as pale-grey needles.

### II) Synthesis of Amine Precursors

### a) Synthesis of 2-(2-dimethylaminoethoxy)-5-methylphenylamine 2

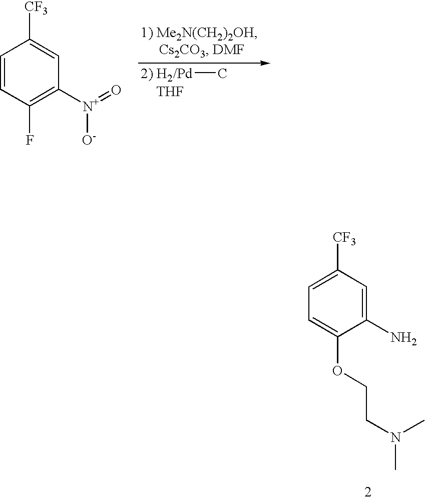

6.5 ml (30 mmol) of 4-fluoro-3-nitrobenzotrifluoride is dissolved in dimethylformamide, 1.3 eq. of 2-dimethylaminoethanol and 2.5 eq of caesium carbonate are added, and the mixture is stirred at 70° C. for 2 hours. The reaction mixture is filtered with suction, and the filtrate is evaporated. The residue is taken up in ethyl acetate, washed a number of times with water, dried over Na₂SO₄, filtered and subsequently evaporated to dryness. The residue is purified by means of column chromatography (100% petroleum ether to 100% ethyl acetate).

Yield: 8.3 g (90%), yellow oil; LC-MS (m/e): 279.2

The nitro compound obtained in this way is hydrogenated for 14 h at room temperature in THF using H₂ and palladium/carbon. The catalyst is filtered off, and the filtrate is evaporated to dryness. The residue is purified by means of column chromatography (dichloromethane/methanol 9:1) to give 2.

Yield: 5.76 g (77%) of 2, pale-grey crystals, LC-MS (m/e): 249.2; HPLC: 0.75 min.

### b) Synthesis of 5-chloro-2-methoxy-4-methylphenylamine 3

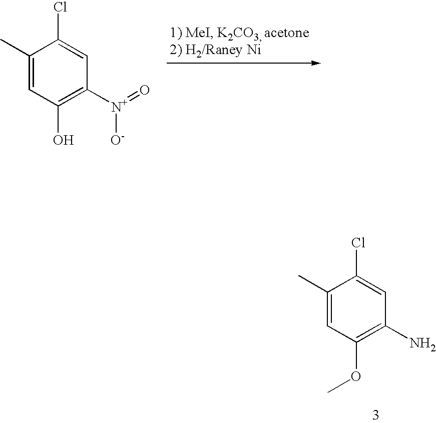

4-Chloro-6-nitro-m-cresol is dissolved in acetone, K₂CO₃ (1 eq.) and iodomethane (1 eq) are added, and the mixture is refluxed overnight. The reaction mixture is filtered, and the filtrate is evaporated to dryness. The red residue is taken up in ethyl acetate, washed with water and NaHCO₃ solution. The org. phase is dried over Na₂SO₄, filtered and evaporated to dryness.

Yield: 7.6 g (45%) of orange solid; LC-MS (m/e): 202.

This compound is hydrogenated for 1 h at room temperature in THF using H₂ and Raney Ni. The catalyst is filtered off, and the filtrate is evaporated to dryness.

Yield: 5.3 g (81%) of 3, brown solid; LC-MS (m/e): 172.

### c) Synthesis of 4-chloro-2-(2-dimethylaminoethoxy)-5-methylphenylamine 3a

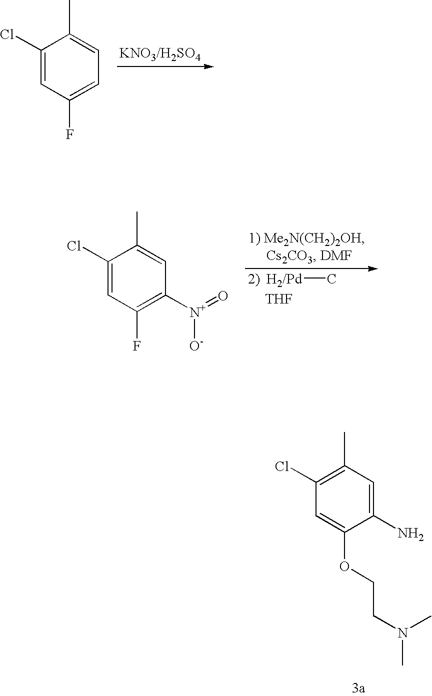

Potassium nitrate (1.1 eq) is added at 0° C. to 2-chloro-4-fluorotoluene (15 ml) in conc. sulfuric acid (200 ml) and allowed to come to room temperature after stirring for 10 min. The reaction mixture is added to ice-water and extracted with ethyl acetate. The combined organic phases are washed with water and saturated sodium chloride solution, dried over sodium sulfate, and the solvent is removed in vacuo. The residue is purified by means of column chromatography (dichloromethane/pentane 1:9). Yield: 8.8 g (37%) of brown crystals.

32 mmol of 4-fluoro-3-nitrobenzotrifluoride are dissolved in dimethylformamide, 1.3 eq. of 2-dimethylaminoethanol and 2.5 eq of caesium carbonate are added, and the mixture is stirred overnight at 50° C. The reaction mixture is filtered with suction, and the filtrate is evaporated. The residue is taken up in ethyl acetate, washed a number of times with water, dried over sodium sulfate, filtered and subsequently evaporated to dryness.

Yield: 6.9 g (77%), yellow oil

The nitro compound obtained in this way is hydrogenated for 14 h at room temperature in THF using H₂ and palladium/carbon. The catalyst is filtered off, and the filtrate is evaporated to dryness.

Yield: 5.7 g (100%) of 3a, brown crystals

### d) Synthesis of tert-butyl 4-(2-amino-5-chloro-4-methylphenoxy)-piperidine-1-carboxylate 3b

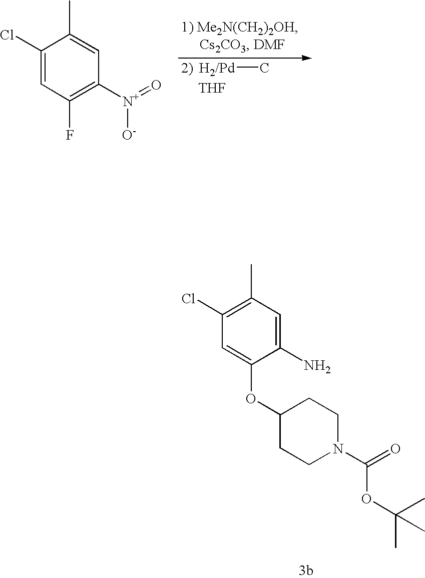

2.6 mmol of 4-fluoro-3-nitrobenzotrifluoride are dissolved in dimethylformamide, 1.1 eq. of tBu 4-hydroxy-1-piperidinecarboxylate and 2.5 eq of caesium carbonate are added, and the mixture is stirred overnight at 50° C. The reaction mixture is filtered with suction, and the filtrate is evaporated. The residue is taken up in ethyl acetate, washed a number of times with water, dried over sodium sulfate, filtered and subsequently evaporated to dryness.

Yield: 1.1 g (99%), brown oil

The nitro compound obtained in this way is hydrogenated for 14 h at room temperature in THF using H₂ and Raney nickel. The catalyst is filtered off, and the filtrate is evaporated to dryness.

Yield: 1.0 g (100%) of 3b, brown oil

### III) Synthesis of the Compounds of the General Formula I

a) Aniline Coupling

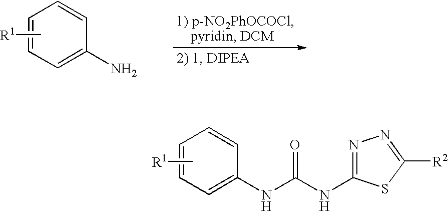

Aniline 2, 3 or commercial amine (1 eq) is dissolved in dichloromethane together with 4-nitrophenyl chloroformate (1.1 eq), pyridine (1 eq) is added at room temperature, and the mixture is stirred for 2 hours. A solution of aminothiadiazole (1a, 1b, 1c or 1d, 1 eq) in dichloromethane is subsequently added, and N-ethyidiisopropylamine (1 eq) is added dropwise, and the mixture is stirred overnight at room temperature. The resultant precipitate is filtered off, washed with dichloromethane and dried overnight at 50° C. and 100 mbar. If necessary, the compound is recrystallised or purified by column chromatography.

Substitution pattern, yield and analysis of the compounds 4 to 8 are given in Example 1.
b) Isocyanate Coupling

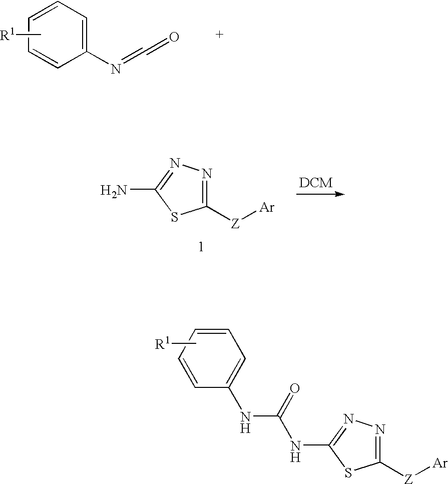

The corresponding isocyanate (1.1 eq) in dichloromethane is added dropwise to a solution of thiadiazolamine (1a, 1b, 1c or 1d; 1 eq) in dichloromethane. The reaction mixture is stirred overnight at room temperature. The resultant precipitate is filtered off, washed with dichloromethane and dried overnight at 50° C. at 100 mbar. If necessary, the compound is recrystallised or purified by column chromatography.

### IV) Removal of Protecting Groups

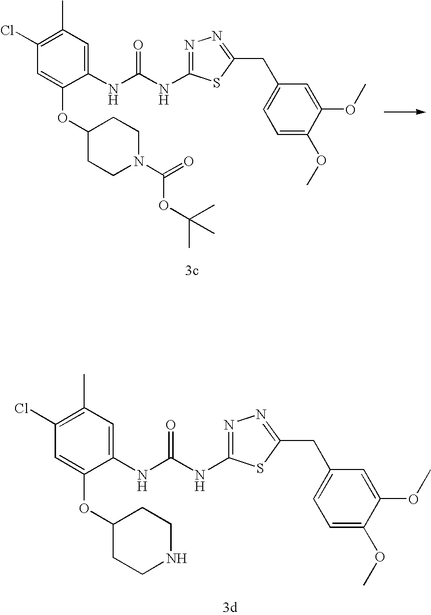

Compound 3c (23 mg, prepared by method IIIa) is dissolved in dichloromethane, trifluoroacetic acid (60 eq) is added, and the mixture is stirred at room temperature for 1 h. The solvent is subsequently removed under reduced pressure. The residue is taken up with dichloromethane and extracted with 1N NaOH and water. The organic phase is dried over sodium sulfate, and the solvent is removed under reduced pressure.

Yield: 11 mg (53%) of 3d white solid (1-[4-chloro-5-methyl-2-(piperidin-4-yloxy)phenyl]-3-[5-(3,4-dimethoxybenzyl)-1,3,4-thiadiazol-2-yl]urea).

V) Enantiomer Separation:

Column-chromatographic separations of individual products formed as racemates into their enantiomers are carried out by the following methods:
a) The substance to be separated is separated via a Hibar 25×5 cm Chiralcel OJ with ethanol. The fractions obtained are separated once more via the said column.
b) The substance to be separated is separated via a Hibar 25×5 cm Chiralcel OJ with ethanol.
c) The substance to be separated is separated via a Hibar 25×5 cm Chiralpak AD with methanol.

### EXAMPLE 1

The following compounds are prepared analogously to the synthetic process in accordance with III a):

with 2-methoxy-5-trifluoromethylaniline and compound 1c 1-(2-methoxy-5-trifluoromethylphenyl)-3-(5-pyridin-4-ylmethyl-1,3,4-thiadiazol-2-yl)-urea 4

with compound 3 and compound 1a 1-(5-chloro-2-methoxy-4-methylphenyl)-3-[5-(3,4-dimethoxybenzyl)-1,3,4-thiadiazol-2-yl]urea 5

with 3-trifluoromethoxyaniline and compound 1a 1-[5-(3,4-dimethoxybenzyl)-1,3,4-thiadiazol-2-yl]-3-(3-trifluoromethoxyphenyl)urea 6

with 3-trifluoromethanesulfonylaniline and compound 1b 1-[5-(1-phenylethyl)-1,3,4-thiadiazol-2-yl]-3-(3-trifluoromethanesulfonylphenyl)urea 7

with 2-methoxy-5-trifluoromethylaniline and compound 1a 1-[5-(3,4-dimethoxybenzyl)-1,3,4-thiadiazol-2-yl]-3-(2-methoxy-5-trifluoromethylphenyl)urea 8

### EXAMPLE 2

The following compounds are prepared analogously to the synthetic process in accordance with III b):

with 4-methylphenyl isocyanate and compound 1b 1-[5-(1-phenylethyl)-1,3,4-thiadiazol-2-yl]-3-p-tolylurea 9

with 3-chlorophenyl isocyanate and compound 1c 1-(3-chlorophenyl)-3-(5-pyridin-4-ylmethyl-1,3,4-thiadiazol-2-yl)urea 10

with 3-chlorophenyl isocyanate and compound 1d 1-(3-chlorophenyl)-3-(5-pyridin-2-ylmethyl-1,3,4-thiadiazol-2-yl)urea 11

with 2-methoxyphenyl isocyanate and compound 1b 1-(2-methoxyphenyl)-3-[5-(1-phenylethyl)-1,3,4-thiadiazol-2-yl]urea 12

with 4-methoxyphenyl isocyanate and compound 1b 1-(4-methoxyphenyl)-3-[5-(1-phenylethyl)-1,3,4-thiadiazol-2-yl]urea 13

with 4-chlorophenyl isocyanate and compound 1b 1-(4-chlorophenyl)-3-[5-(1-phenylethyl)-1,3,4-thiadiazol-2-yl]urea 14

with 3-chlorophenyl isocyanate and compound 1b 1-(3-chlorophenyl)-3-[5-(1-phenylethyl)-1,3,4-thiadiazol-2-yl]urea 15

with 3-chloro-4-methylphenyl isocyanate and compound 1c 1-(3-chloro-4-methylphenyl)-3-(5-pyridin-4-ylmethyl-1,3,4-thiadiazol-2-yl)urea 16

with 2-methoxy-5-methylphenyl isocyanate and compound 1b 1-(2-methoxy-5-methylphenyl)-3-[5-(1-phenylethyl)-1,3,4-thiadiazol-2-yl]urea 17

with 3-chloro-4-methylphenyl isocyanate and compound and compound 1b 1-(3-chloro-4-methylphenyl)-3-[5-(1-phenylethyl)-1,3,4-thiadiazol-2-yl]urea 18

with 3-chloro-5-methylphenyl isocyanate and compound 1b 1-(5-chloro-2-methylphenyl)-3-[5-(1-phenylethyl)-1,3,4-thiadiazol-2-yl]urea 19

with 3-chloro-2-methylphenyl isocyanate and compound 1b 1-(3-chloro-2-methylphenyl)-3-[5-(1-phenylethyl)-1,3,4-thiadiazol-2-yl]urea 20

with 3-chloro-5-methoxyphenyl isocyanate and compound 1c 1-(5-chloro-2-methoxyphenyl)-3-(5-pyridin-4-ylmethyl-1,3,4-thiadiazol-2-yl)urea 21

with 3-chloro-5-methoxyphenyl isocyanate and compound 1d 1-(5-chloro-2-methoxyphenyl)-3-(5-pyridin-2-ylmethyl-1,3,4-thiadiazol-2-yl)urea 22

with 3-trifluoromethylphenyl isocyanate and compound 1d 1-(5-pyridin-2-ylmethyl-1,3,4-thiadiazol-2-yl)-3-(3-trifluoromethylphenyl)urea 23

with 3-trifluoromethylphenyl isocyanate and compound 1c 1-(5-pyridin-4-ylmethyl-1,3,4-thiadiazol-2-yl)-3-(3-trifluoromethylphenyl)urea 24

with 4-methylphenyl isocyanate and compound 1a 1-[5-(3,4-dimethoxybenzyl)-1,3,4-thiadiazol-2-yl]-3-p-tolylurea 25

with 5-chloro-3-methoxyphenyl isocyanate and compound 1b 1-(5-chloro-2-methoxyphenyl)-3-[5-(1-phenylethyl)-1,3,4-thiadiazol-2-yl]urea 26

with 3,4-dichlorophenyl isocyanate and compound 1b 1-(3,4-dichlorophenyl)-3-[5-(1-phenylethyl)-1,3,4-thiadiazol-2-yl]urea 27

with 3-trifluoromethylphenyl isocyanate and compound 1b 1-[5-(1-phenylethyl)-1,3,4-thiadiazol-2-yl]-3-(3-trifluoromethylphenyl)urea 28

with 4-trifluoromethylphenyl isocyanate and compound 1b 1-[5-(1-phenylethyl)-1,3,4-thiadiazol-2-yl]-3-(4-trifluoromethylphenyl)urea 29

with 2,3-dichlorophenyl isocyanate and compound 1b 1-(2,3-dichlorophenyl)-3-[5-(1-phenylethyl)-1,3,4-thiadiazol-2-yl]urea 30

with 2-methoxyphenyl isocyanate and compound 1a 1-[5-(3,4-dimethoxybenzyl)-1,3,4-thiadiazol-2-yl]-3-(2-methoxyphenyl)urea 31

with 4-chlorophenyl isocyanate and compound 1a 1-(4-chlorophenyl)-3-[5-(3,4-dimethoxybenzyl)-1,3,4-thiadiazol-2-yl]urea 32

with 3-chlorophenyl isocyanate and compound 1a 1-(3-chlorophenyl)-3-[5-(3,4-dimethoxybenzyl)-1,3,4-thiadiazol-2-yl]urea 33

with 4-trifluoromethoxyphenyl isocyanate and compound 1b 1-[5-(1-phenylethyl)-1,3,4-thiadiazol-2-yl]-3-(4-trifluoromethoxyphenyl)urea 34

with 4-flouro-3-trifluoromethylphenyl isocyanate and compound 1b 1-(4-fluoro-3-trifluoromethylphenyl)-3-[5-(1-phenylethyl)-1,3,4-thiadiazol-2-yl]urea 35

with 2-methoxy-5-methylphenyl isocyanate and compound 1a 1-[5-(3,4-dimethoxybenzyl)-1,3,4-thiadiazol-2-yl]-3-(2-methoxy-5-methylphenyl)-urea 36

with 3-chloro-2-methylphenyl isocyanate and compound 1a 1-(3-chloro-2-methylphenyl)-3-[5-(3,4-dimethoxybenzyl)-1,3,4-thiadiazol-2-yl]urea 37

with 5-chloro-2-methylphenyl isocyanate and compound 1al-(5-chloro-2-methylphenyl)-3-[5-(3,4-dimethoxybenzyl)-1,3,4-thiadiazol-2-yl]urea 38

with 3-chloro-5-methylphenyl isocyanate and compound 1a 1-(3-chloro-4-methylphenyl)-3-[5-(3,4-dimethoxybenzyl)-1,3,4-thiadiazol-2-yl]urea 39

with 4-chloro-3-trifluoromethylphenyl isocyanate and compound 1b 1-(4-chloro-3-trifluoromethylphenyl)-3-[5-(1-phenylethyl)-1,3,4-thiadiazol-2-yl]urea 40

with 2,5-dimethoxyphenyl isocyanate and compound 1a 1-[5-(3,4-dimethoxybenzyl)-1,3,4-thiadiazol-2-yl]-3-(2,5-dimethoxyphenyl)urea 41

2,4-dimethoxyphenyl isocyanate and compound 1a 1-[5-(3,4-dimethoxybenzyl)-1,3,4-thiadiazol-2-yl]-3-(2,4-dimethoxyphenyl)urea 42

with 5-chloro-2-methoxyphenyl isocyanate and compound 1a 1-(5-chloro-2-methoxyphenyl)-3-[5-(3,4-dimethoxybenzyl)-1,3,4-thiadiazol-2-yl]urea 43

with 3-chloro-4-methoxyphenyl isocyanate and compound 1a 1-(3-chloro-4-methoxyphenyl)-3-[5-(3,4-dimethoxybenzyl)-1,3,4-thiadiazol-2-yl]urea 44

with 3-trifluoromethylphenyl isocyanate and compound 1a 1-[5-(3,4-dimethoxybenzyl)-1,3,4-thiadiazol-2-yl]-3-(3-trifluoromethylphenyl)urea 45

with 3,4-dichlorophenyl isocyanate and compound 1a 1-(3,4-dichlorophenyl)-3-[5-(3,4-dimethoxybenzyl)-1,3,4-thiadiazol-2-yl]urea 46

with 4-trifluoromethylphenyl isocyanate and compound 1a 1-[5-(3,4-dimethoxybenzyl)-1,3,4-thiadiazol-2-yl]-3-(4-trifluoromethylphenyl)urea 47

with 2,3-dichlorophenyl isocyanate and compound 1a 1-(2,3-dichlorophenyl)-3-[5-(3,4-dimethoxybenzyl)-1,3,4-thiadiazol-2-yl]urea 48

with 4-trifluoromethoxyphenyl isocyanate and compound 1a 1-[5-(2,3-dimethoxybenzyl)-1,3,4-thiadiazol-2-yl]-3-(4-trifluoromethoxyphenyl)urea 49

with 2-trifluoromethoxyphenyl isocyanate and compound 1a 1-[5-(2,3-dimethoxybenzyl)-1,3,4-thiadiazol-2-yl]-3-(2-trifluoromethoxyphenyl)urea 50

with 4-fluoro-3-trifluoromethylphenyl isocyanate and compound 1a 1-[5-(3,4-dimethoxybenzyl)-1,3,4-thiadiazol-2-yl]-3-(4-fluoro-3-trifluoromethylphenyl)urea 51

with 5-chloro-2,4-dimethoxyphenyl isocyanate and compound 1a 1-(5-chloro-2,4-dimethoxyphenyl)-3-[5-(3,4-dimethoxybenzyl)-1,3,4-thiadiazol-2-yl]urea 52

with 4-chloro-3-trifluoromethylphenyl isocyanate and compound 1a 1-(4-chloro-3-trifluoromethylphenyl)-3-[5-(3,4-dimethoxybenzyl)-1,3,4-thiadiazol-2-yl]urea 53

with 2,4-dimethoxyphenyl isocyanate and compound 1b 1-(2,4-dimethoxyphenyl)-3-[5-(1-phenylethyl)-1,3,4-thiadiazol-2-yl]urea 54

with 3-chloro-4-methoxyphenyl isocyanate and compound 1b 1-(3-chloro-4-methoxyphenyl)-3-[5-(1-phenylethyl)-1,3,4-thiadiazol-2-yl]urea 55

with 2-(2-dimethylaminoethoxy)phenyl isocyanate 2 and compound 1b 1-[2-(2-dimethylaminoethoxy)-5-trifluoromethylphenyl]-3-[5-(1-phenylethyl)-1,3,4-thiadiazol-2-yl]urea 56

### EXAMPLE 3

The following compounds are prepared analogously to the synthetic process in accordance with III a):

with 2-methoxy-5-trifluoromethylaniline and compound 1b 1-(2-methoxy-5-trifluoromethylphenyl)-3-[5-(1-phenylethyl)-1,3,4-thiadiazol-2-yl]urea 57

with 5-chloro-2-methoxy-4-methylaniline and compound 1c 1-(5-chloro-2-methoxy-4-methylphenyl)-3-(5-pyridin-4-ylmethyl-1,3,4-thiadiazol-2-yl)urea 58

with 3-trifluoromethoxyaniline and compound 1c 1-(5-pyridin-4-ylmethyl-1,3,4-thiadiazol-2-yl)-3-(3-trifluoromethoxyphenyl)urea 59

with 5-chloro-2-methoxy-4-methylaniline and compound 1b 1-(5-chloro-2-methoxy-4-methylphenyl)-3-[5-(1-phenylethyl)-1,3,4-thiadiazol-2-yl]urea 60

with 3-trifluoromethoxyaniline and compound 1b 1-[5-(1-phenylethyl)-1,3,4-thiadiazol-2-yl]-3-(3-trifluoromethoxyphenyl)urea 61

with 2-methoxy-5-trifluoromethylaniline and compound 1b 1-(2-methoxy-5-trifluoromethylphenyl)-3-[5-(1-phenylpropyl)-1,3,4-thiadiazol-2-yl]urea 62

with 5-chloro-2-methoxy-4-methylaniline and 1e 1-(5-chloro-2-methoxy-4-methylphenyl)-3-[5-(4-chlorophenoxymethyl)-1,3,4-thiadiazol-2-yl]urea 63

with 3-trifluoromethoxyaniline and 1e 1-[5-(4-chlorophenoxymethyl)-1,3,4-thiadiazol-2-yl]-3-(3-trifluoromethoxyphenyl)urea 64

with compound 3a and compound 1b 1-[4-chloro-2-(2-dimethylaminoethoxy)-5-methylphenyl]-3-[5-(1-phenylethyl)-1,3,4-thiadiazol-2-yl]urea 65

with compound 3a and compound 1a 1-[4-chloro-2-(2-dimethylaminoethoxy)-5-methylphenyl]-3-[5-(3,4-dimethoxybenzyl)-1,3,4-thiadiazol-2-yl]urea 66)

with compound 2 and compound 1a 1-[5-(3,4-dimethoxybenzyl)-1,3,4-thiadiazol-2-yl]-3-[2-(2-dimethylaminoethoxy)-5-trifluoromethylphenyl]urea 67

### EXAMPLE 4

The following compounds are prepared analogously to the synthetic process in accordance with III b):

with 2-methoxy-5-methylaniline and compound 1g 1-(2-methoxy-5-methylphenyl)-3-[5-(1-phenylpropyl)-1,3,4-thiadiazol-2-yl]urea 68

with 2,5-dimethoxyaniline and compound 1b 1-(2,5-dimethoxyphenyl)-3-[5-(1-phenylethyl)-1,3,4-thiadiazol-2-yl]urea 69

with 3-chloro-4-methylaniline and compound 1g 1-(3-chloro-4-methylphenyl)-3-[5-(1-phenylpropyl)-1,3,4-thiadiazol-2-yl]urea 70

with 2,5-dichloroaniline and compound 1b 1-(2,5-dichlorophenyl)-3-[5-(1-phenylethyl)-1,3,4-thiadiazol-2-yl]urea 71

with 3-trifluoromethylaniline and compound 1f 1-[5-(hydroxyphenylmethyl)-1,3,4-thiadiazol-2-yl]-3-(3-trifluoromethylphenyl)urea 72

with 2-methoxy-5-methylaniline and compound 1h 1-(2-methoxy-5-methylphenyl)-3-[5-(2-methyl-1-phenylpropyl)-1,3,4-thiadiazol-2-yl]urea 73

with 2-fluoro-5-trifluoromethylaniline and compound 1c 1-(2-fluoro-5-trifluoromethylphenyl)-3-(5-pyridin-4-ylmethyl-1,3,4-thiadiazol-2-yl)urea 74

with 4-fluoro-3-trifluoromethylaniline and compound 1c 1-(4-fluoro-3-trifluoromethylphenyl)-3-(5-pyridin-4-ylmethyl-1,3,4-thiadiazol-2-yl)urea 75

with 3-methylaniline and compound 1i 1-[5-(3,4-dimethoxybenzoyl)-1,3,4-thiadiazol-2-yl]-3-m-tolylurea 76

with 3-methylaniline and compound 1k 1-{5-[2-(3,4-dimethoxyphenyl)ethyl]-1,3,4-thiadiazol-2-yl}-3-m-tolylurea 77

with 3-chloro-4-methylaniline and compound 1h 1-(3-chloro-4-methylphenyl)-3-[5-(2-methyl-1-phenylpropyl)-1,3,4-thiadiazol-2-yl]urea 78

with 3-chloroaniline and compound 1j 1-(3-chlorophenyl)-3-[5-(3,4-dimethoxyphenoxy)-1,3,4-thiadiazol-2-yl]urea 79

with 3-chloroaniline and compound 1i 1-(3-chlorophenyl)-3-[5-(3,4-dimethoxybenzoyl)-1,3,4-thiadiazol-2-yl]urea 80

with 3-chloroaniline and compound 1k 1-(3-chlorophenyl)-3-{5-[2-(3,4-dimethoxyphenyl)ethyl]-1,3,4-thiadiazol-2-yl}urea 81

with 5-chloro-2,4-dimethoxyaniline and compound 1b 1-(5-chloro-2,4-dimethoxyphenyl)-3-[5-(1-phenylethyl)-1,3,4-thiadiazol-2-yl]urea 82

with 3-chloroaniline and compound 1l 1-(3-chlorophenyl)-3-[5-(3,4-dimethoxybenzylamino)-1,3,4-thiadiazol-2-yl]urea 83

with 3-trifluoromethylaniline and compound 1m 1-[5-(3,4-dimethoxyphenylamino)-1,3,4-thiadiazol-2-yl]-3-(3-trifluoromethylphenyl)urea 84

with 3-trifluoromethylaniline and compound 1j 1-[5-(3,4-dimethoxyphenoxy)-1,3,4-thiadiazol-2-yl]-3-(3-trifluoromethylphenyl)urea 85

with 4-fluoro-3-trifluoromethylaniline and compound 1e 1-[5-(4-chlorophenoxymethyl)-1,3,4-thiadiazol-2-yl]-3-(4-fluoro-3-trifluoromethylphenyl)-urea 86

with 5-chloro-2-methoxyaniline and compound 1k 1-(5-chloro-2-methoxyphenyl)-3-{5-[2-(3,4-dimethoxyphenyl)ethyl]-1,3,4-thiadiazol-2-yl}urea 87

with 5-chloro-2-methoxyaniline and compound 1i 1-(5-chloro-2-methoxyphenyl)-3-[5-(3,4-dimethoxybenzoyl)-1,3,4-thiadiazol-2-yl]urea 88

with 5-chloro-2-methoxyaniline and compound 1l 1-(5-chloro-2-methoxyphenyl)-3-[5-(3,4-dimethoxybenzylamino)-1,3,4-thiadiazol-2-yl]urea 89

with 3-trifluoromethylaniline and compound 1k 1-{5-[2-(3,4-dimethoxyphenyl)ethyl]-1,3,4-thiadiazol-2-yl}-3-(3-trifluoromethylphenyl)-urea 90

with 3-trifluoromethylaniline and compound 1l 1-[5-(3,4-dimethoxybenzylamino)-1,3,4-thiadiazol-2-yl]-3-(3-trifluoromethylphenyl)urea 91

with 2-fluoro-3-trifluoromethylaniline and compound 1m 1-[5-(3,4-dimethoxyphenylamino)-1,3,4-thiadiazol-2-yl]-3-(2-fluoro-3-trifluoromethyl-phenyl)urea 92

with 2-fluoro-3-trifluoromethylaniline and compound 1j 1-[5-(3,4-dimethoxyphenoxy)-1,3,4-thiadiazol-2-yl]-3-(2-fluoro-3-trifluoromethylphenyl)-urea 93

with 4-fluoro-3-trifluoromethylaniline and compound 1j 1-[5-(3,4-dimethoxyphenoxy)-1,3,4-thiadiazol-2-yl]-3-(4-fluoro-3-trifluoromethylphenyl)-urea 94

with 3-fluoro-5-trifluoromethylaniline and compound 1i 1-[5-(3,4-dimethoxybenzoyl)-1,3,4-thiadiazol-2-yl]-3-(3-fluoro-5-trifluoromethylphenyl)-urea 95

with 3-fluoro-5-trifluoromethylaniline and compound 1k 1-{5-[2-(3,4-dimethoxyphenyl)ethyl]-1,3,4-thiadiazol-2-yl}-3-(3-fluoro-5-trifluoromethylphenyl)urea 96

with 2-fluoro-5-trifluoromethylaniline and compound 1i 1-[5-(3,4-dimethoxybenzoyl)-1,3,4-thiadiazol-2-yl]-3-(2-fluoro-5-trifluoromethylphenyl)-urea 97

with 4-fluoro-3-trifluoromethylaniline and compound 1i 1-[5-(3,4-dimethoxybenzoyl)-1,3,4-thiadiazol-2-yl]-3-(4-fluoro-3-trifluoromethylphenyl)-urea 98

with 2-fluoro-3-trifluoromethylaniline and compound 1i 1-[5-(3,4-dimethoxybenzoyl)-1,3,4-thiadiazol-2-yl]-3-(2-fluoro-3-trifluoromethylphenyl)-urea 99

with 4-fluoro-3-trifluoromethylaniline and compound 1k 1-{5-[2-(3,4-dimethoxyphenyl)ethyl]-1,3,4-thiadiazol-2-yl}-3-(4-fluoro-3-trifluoromethylphenyl)urea 100

with 2-fluoro-3-trifluoromethylaniline and compound 1k 1-{5-[2-(3,4-dimethoxyphenyl)ethyl]-1,3,4-thiadiazol-2-yl}-3-(2-fluoro-3-trifluoromethylphenyl)urea 101

with 2-fluoro-5-trifluoromethylaniline and compound 1k 1-{5-[2-(3,4-dimethoxyphenyl)ethyl]-1,3,4-thiadiazol-2-yl}-3-(2-fluoro-5-trifluoromethylphenyl)urea 102

with 2-fluoro-3-trifluoromethylaniline and compound 1l 1-[5-(3,4-dimethoxybenzylamino)-1,3,4-thiadiazol-2-yl]-3-(2-fluoro-3-trifluoromethylphenyl)urea 103

with 4-chloro-3-trifluoromethylaniline and compound 1k 1-(4-chloro-3-trifluoromethylphenyl)-3-{5-[2-(3,4-dimethoxyphenyl)ethyl]-1,3,4-thiadiazol-2-yl}urea 104

with 4-chloro-3-trifluoromethylaniline and compound 1i 1-(4-chloro-3-trifluoromethylphenyl)-3-[5-(3,4-dimethoxybenzoyl)-1,3,4-thiadiazol-2-yl]urea 105

with 4-chloro-3-trifluoromethylaniline and compound 1l 1-(4-chloro-3-trifluoromethylphenyl)-3-[5-(3,4-dimethoxybenzylamino)-1,3,4-thiadiazol-2-yl]urea 106

with 3,5-bistrifluoromethylaniline and compound 1m 1-(3,5-bistrifluoromethylphenyl)-3-[5-(3,4-dimethoxyphenylamino)-1,3,4-thiadiazol-2-yl]urea 107

with 3,5-bistrifluoromethylaniline and compound 1i 1-(3,5-bistrifluoromethylphenyl)-3-[5-(3,4-dimethoxybenzoyl)-1,3,4-thiadiazol-2-yl]urea 108

with 3,5-bistrifluoromethylaniline and compound 1k 1-(3,5-bistrifluoromethylphenyl)-3-{5-[2-(3,4-dimethoxyphenyl)ethyl]-1,3,4-thiadiazol-2-yl}-urea 109

with 3,5-bistrifluoromethylaniline and compound 1l 1-(3,5-bistrifluoromethylphenyl)-3-[5-(3,4-dimethoxybenzylamino)-1,3,4-thiadiazol-2-yl]urea 110

with 3-chloroaniline and compound 1n 1-(3-chlorophenyl)-3-[5-(pyridin-4-yloxy)-1,3,4-thiadiazol-2-yl]urea 111

with 3-trifluoromethylaniline and compound 1n 1-[5-(pyridin-4-yloxy)-1,3,4-thiadiazol-2-yl]-3-(3-trifluoromethylphenyl)urea 112

with 4-fluoro-3-trifluoromethylaniline and compound 1n 1-(4-fluoro-3-trifluoromethylphenyl)-3-[5-(pyridin-4-yloxy)-1,3,4-thiadiazol-2-yl]urea 113

with 2-fluoro-3-trifluoromethylaniline and compound 1n 1-(2-fluoro-3-trifluoromethylphenyl)-3-[5-(pyridin-4-yloxy)-1,3,4-thiadiazol-2-yl]urea 114

with 2-fluoro-5-trifluoromethylaniline and compound 1n 1-(2-fluoro-5-trifluoromethylphenyl)-3-[5-(pyridin-4-yloxy)-1,3,4-thiadiazol-2-yl]urea 115

with 3,5-bistrifluoromethylaniline and compound 1n 1-(3,5-bistrifluoromethylphenyl)-3-[5-(pyridin-4-yloxy)-1,3,4-thiadiazol-2-yl]urea 116

with 5-chloro-2-methoxyaniline and compound 1e 1-(5-chloro-2-methoxyphenyl)-3-[5-(4-chlorophenoxymethyl)-1,3,4-thiadiazol-2-yl]urea 117

with 3-trifluoromethylaniline and compound 1e 1-[5-(4-chlorophenoxymethyl)-1,3,4-thiadiazol-2-yl]-3-(3-trifluoromethylphenyl)urea 118

with 3-trifluoromethylaniline and compound 1i 1-[5-(3,4-dimethoxybenzoyl)-1,3,4-thiadiazol-2-yl]-3-(3-trifluoromethylphenyl)urea 119 (EMD521745

with 3-methylaniline and compound 1o 1-[5-(3,4-dimethoxyphenoxymethyl)-1,3,4-thiadiazol-2-yl]-3-m-tolylurea 120

with 3-chloroaniline and compound 1o 1-(3-chlorophenyl)-3-[5-(3,4-dimethoxyphenoxymethyl)-1,3,4-thiadiazol-2-yl]urea 121

with 5-chloro-2-methoxyaniline and compound 1o 1-(5-chloro-2-methoxyphenyl)-3-[5-(3,4-dimethoxyphenoxymethyl)-1,3,4-thiadiazol-2-yl]urea 122

with 3-trifluoromethylaniline and compound 1o 1-[5-(3,4-dimethoxyphenoxymethyl)-1,3,4-thiadiazol-2-yl]-3-(3-trifluoromethylphenyl)urea 123

with 2-fluoro-3-trifluoromethylaniline and compound 1o 1-[5-(3,4-dimethoxyphenoxymethyl)-1,3,4-thiadiazol-2-yl]-3-(2-fluoro-3-trifluoromethylphenyl)urea 124

with 3-fluoro-5-trifluoromethylaniline and compound 1o 1-[5-(3,4-dimethoxyphenoxymethyl)-1,3,4-thiadiazol-2-yl]-3-(3-fluoro-5-trifluoromethylphenyl)urea 125

with 4-fluoro-3-trifluoromethylaniline and compound 1o 1-[5-(3,4-dimethoxyphenoxymethyl)-1,3,4-thiadiazol-2-yl]-3-(4-fluoro-3-trifluoromethylphenyl)urea 126

with 2-fluoro-5-trifluoromethylaniline and compound 1o 1-[5-(3,4-dimethoxyphenoxymethyl)-1,3,4-thiadiazol-2-yl]-3-(2-fluoro-5-trifluoromethylphenyl)urea 127

with 4-chloro-3-trifluoromethylaniline and compound 1o 1-(4-chloro-3-trifluoromethylphenyl)-3-[5-(3,4-dimethoxyphenoxymethyl)-1,3,4-thiadiazol-2-yl]urea 128

with 3,5-bistrifluoromethylaniline and compound 1o 1-(3,5-bistrifluoromethylphenyl)-3-[5-(3,4-dimethoxyphenoxymethyl)-1,3,4-thiadiazol-2-yl]urea 129

### EXAMPLE 5

Preparation of 1-[4-chloro-5-methyl-2-(piperidin-4-yloxy)phenyl]-3-[5-(3,4-dimethoxybenzyl)-1,3,4-thiadiazol-2-yl]urea 3d.

3c is prepared analogously to the synthetic process in accordance with III b using 3b and 1a. 3c is subsequently converted into 3d by process IV.

### EXAMPLE 6

Compound 28 is separated into
(S)-1-[5-(1-phenylethyl)-1,3,4-thiadiazol-2-yl]-3-(3-trifluoromethylphenyl)-urea 130 and
(R)-1-[5-(1-phenylethyl)-1,3,4-thiadiazol-2-yl]-3-(3-trifluoromethylphenyl)-urea 131
   by process V a).

Compound 26 is separated into
(S)-1-(5-chloro-2-methoxyphenyl)-3-[5-(1-phenylethyl)-1,3,4-thiadiazol-2-yl]urea enantiomer 132 and
(R)-1-(5-chloro-2-methoxyphenyl)-3-[5-(1-phenylethyl)-1,3,4-thiadiazol-2-yl]urea 133
   by process V b).

Compound 35 is separated into
(S)-1-(4-fluoro-3-trifluoromethylphenyl)-3-[5-(1-phenylethyl)-1,3,4-thiadiazol-2-yl]urea 134 and
(R)-1-(4-fluoro-3-trifluoromethylphenyl)-3-[5-(1-phenylethyl)-1,3,4-thiadiazol-2-yl]urea 135
   by process V c).

The analytical characteristic data of the compounds are shown in Table 1:

TABLE 1

| | Retention time | LC-MS/ | HPLC |
|---|---|---|---|
| Molecule | HPLC/min | m/e | method |
| 4 | 2.51 | 410.2 | 1 |
| 5 | 3.35 | 449.2 | 1 |
| 6 | 3.23 | 455.2 | 1 |
| 7 | 3.23 | 457.2 | 1 |
| 8 | 3.24 | 469.2 | 1 |
| 9 | 3.37 | 339.2 | 1 |
| 10 | 2.73 | 346.2 | 1 |
| 11 | 2.74 | 346.2 | 1 |
| 12 | 3.36 | 355.2 | 1 |
| 13 | 3.29 | 355.2 | 1 |
| 14 | 3.48 | 359.2 | 1 |
| 15 | 3.47 | 359.2 | 1 |
| 16 | 2.77 | 360.2 | 1 |
| 17 | 3.46 | 369.2 | 1 |
| 18 | 3.53 | 373.2 | 1 |
| 19 | 3.41 | 373.2 | 1 |
| 20 | 3.35 | 373.2 | 1 |
| 21 | 2.76 | 376.2 | 1 |
| 22 | 2.72 | 376.2 | 1 |
| 23 | 2.80 | 380.2 | 1 |
| 24 | 2.76 | 380.2 | 1 |
| 25 | 3.21 | 385.2 | 1 |
| 26 | 3.53 | 389.2 | 1 |
| 27 | 3.61 | 393.2 | 1 |
| 28 | 3.50 | 393.2 | 1 |
| 29 | 3.51 | 393.2 | 1 |
| 30 | 3.33 | 393.2 | 1 |
| 31 | 3.18 | 401.2 | 1 |
| 32 | 3.29 | 405.2 | 1 |
| 33 | 3.31 | 405.2 | 1 |
| 34 | 3.53 | 409.2 | 1 |
| 35 | 3.52 | 411.2 | 1 |
| 36 | 3.31 | 415.2 | 1 |
| 37 | 3.37 | 419.2 | 1 |
| 38 | 3.30 | 419.2 | 1 |
| 39 | 3.12 | 419.2 | 1 |
| 40 | 3.62 | 427.2 | 1 |
| 41 | 3.15 | 431.2 | 1 |
| 42 | 2.61 | 431.2 | 1 |
| 43 | 3.37 | 435.2 | 1 |
| 44 | 3.04 | 435.2 | 1 |
| 45 | 3.35 | 439.2 | 1 |
| 46 | 3.44 | 439.2 | 1 |
| 47 | 3.39 | 439.2 | 1 |
| 48 | 3.41 | 439.2 | 1 |
| 49 | 3.41 | 455.2 | 1 |
| 50 | 3.36 | 455.2 | 1 |
| 51 | 3.39 | 457.2 | 1 |
| 52 | 3.14 | 465.2 | 1 |
| 53 | 3.47 | 473.2 | 1 |
| 54 | 2.88 | 385.2 | 2 |
| 55 | 2.82 | 389.2 | 2 |
| 56 | 2.85 | 408.2 | 2 |
| 57 | 3.13 | 323.3 | 2 |
| 58 | 2.48 | 390.2 | 2 |
| 59 | 2.35 | 396.2 | 2 |
| 60 | 3.27 | 403.2 | 2 |
| 61 | 3.15 | 409.2 | 2 |
| 62 | 3.32 | 437.2 | 2 |
| 63 | 3.36 | 439 | 2 |
| 64 | 3.25 | 445 | 2 |
| 65 | 2.77 | 460.2 | 2 |
| 66 | 2.59 | 506.2 | 2 |
| 67 | 2.61 | 526.3 | 2 |
| 68 | 3.17 | 383.2 | 2 |
| 69 | 2.79 | 385.2 | 2 |
| 70 | 3.26 | 387.2 | 2 |
| 71 | 3.17 | 393.2 | 2 |
| 72 | 2.83 | 395.2 | 2 |
| 73 | 3.23 | 397.2 | 2 |
| 74 | 2.25 | 398 | 2 |
| 75 | 2.34 | 398.2 | 2 |
| 76 | 9.29 | 399 | 6 |
| 77 | 8.54 | 399.1 | 6 |
| 78 | 3.34 | 401.2 | 2 |
| 79 | 7.09 | 406.9 | 6 |
| 80 | 10.05 | 419 | 6 |
| 81 | 6.91 | 419.1 | 6 |
| 82 | 2.95 | 419.2 | 2 |
| 83 | 7.96 | 420.1 | 6 |
| 84 | 8.39 | 440.1 | 6 |
| 85 | 7.15 | 441 | 6 |
| 86 | 3.23 | 447.2 | 2 |
| 87 | 7.52 | 449 | 6 |
| 88 | 10.2 | 449 | 6 |
| 89 | 8.27 | 450.1 | 6 |
| 90 | 7.09 | 453.1 | 6 |
| 91 | 8.07 | 454.1 | 6 |
| 92 | 8.6 | 458.1 | 6 |
| 93 | 7.38 | 459 | 6 |
| 94 | 7.23 | 459 | 6 |
| 95 | 10.65 | 470.9 | 6 |
| 96 | 9.72 | 471 | 6 |
| 97 | 10.64 | 471 | 6 |
| 98 | 10.47 | 471 | 6 |
| 99 | 10.57 | 471 | 6 |
| 100 | 9.2 | 471.1 | 6 |
| 101 | 9.28 | 471.1 | 6 |
| 102 | 9.31 | 471.1 | 6 |
| 103 | 8.32 | 472 | 6 |
| 104 | 8.04 | 487 | 6 |
| 105 | 11.31 | 487 | 6 |
| 106 | 8.78 | 488 | 6 |
| 107 | 8.88 | 508.1 | 6 |
| 108 | 11.91 | 521 | 6 |
| 109 | 8.52 | 521.1 | 6 |
| 110 | 9.36 | 522.1 | 6 |
| 111 | 6.97 | 348 | 6 |
| 112 | 7.1 | 382 | 6 |
| 113 | 7.15 | 400 | 6 |
| 114 | 7.2 | 400 | 6 |
| 115 | 7.25 | 400.2 | 6 |
| 116 | 7.89 | 450.1 | 6 |
| 117 | 3.27 | 425.2 | 2 |
| 118 | 3.21 | 429.2 | 2 |
| 119 | 10.14 | 453 | 6 |
| 120 | 6.42 | 401 | 6 |
| 121 | 6.84 | 421 | 6 |
| 122 | 7.33 | 450.9 | 6 |
| 123 | 6.97 | 454.9 | 6 |
| 124 | 7.29 | 472.8 | 6 |
| 125 | 7.46 | 472.9 | 6 |
| 126 | 7.04 | 473 | 6 |
| 127 | 7.2 | 473 | 6 |
| 128 | 7.77 | 489 | 6 |
| 129 | 8.23 | 523 | 6 |
| 130 | 9.8 | 393.2 | 3 |
| 131 | 13.17 | 393.2 | 3 |
| 132 | 15.25 | 389.2 | 4 |
| 133 | 28.8 | 389.2 | 4 |
| 134 | 9.09 | 411.2 | 5 |
| 135 | 10.64 | 411.2 | 5 |

HPLC method 1: 99% A/1% B for 1 min, to 100% B in 2.5 min and 100% B for 1 min; A: water (0.1% TFA), B: acetonitrile (0.1% TFA); detection at 254 nm; column: Chromolith SpeedRod RP 18

HPLC method 2: 99% A/1% B for 0.5 min, to 100% B in 2.5 min and 100% B for 1 min; A: water (0.1% TFA), B: acetonitrile (0.1% TFA); detection at 254 nm; column: Chromolith SpeedRod RP 18

HPLC method 3: heptane/EtOH 70:30; detection at 254 nm; column: Chiralcel OJ

HPLC method 4: EtOH; detection at 254 nm; column: Chiralcel OJ

HPLC method 5: MeOH; detection at 254 nm; column: Chiralpak AD

HPLC method 6: 80% A/20% B for 2.5 min, to 20% A/80% B in 4 min and 20% A/80% B for 7 min; A: water (0.1% HCOOH), B: acetonitrile (0.1% HCOOH); detection at 254 nm; column: C18 NUCLEODUR (MACHERY NAGEL)

The following examples relate to medicaments:

### EXAMPLE A

### Injection Vials

A solution of 100 g of an active ingredient of the formula I and 5 g of disodium hydrogenphosphate in 3 l of bidistilled water is adjusted to pH 6.5 using 2 N hydrochloric acid, sterile filtered, transferred into injection vials, lyophilised under sterile conditions and sealed under sterile conditions. Each injection vial contains 5 mg of active ingredient.

### EXAMPLE B

### Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### EXAMPLE C

### Solution

A solution is prepared from 1 g of an active ingredient of the formula I, 9.38 g of NaH₂PO₄.2 H₂O, 28.48 g of Na₂HPO₄.12 H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula I are mixed with 99.5 g of Vaseline under aseptic conditions.

### EXAMPLE E

### Tablets

A mixture of 1 kg of active ingredient of the formula I, 4 kg of lactose, 1.2 kg of potato starch, 0.2 kg of talc and 0.1 kg of magnesium stearate is pressed in a conventional manner to give tablets in such a way that each tablet contains 10 mg of active ingredient.

### EXAMPLE F

### Dragees

Tablets are pressed analogously to Example E and subsequently coated in a conventional manner with a coating of sucrose, potato starch, talc, tragacanth and dye.

### EXAMPLE G

### Capsules

2 kg of active ingredient of the formula I are introduced into hard gelatine capsules in a conventional manner in such a way that each capsule contains 20 mg of the active ingredient.

### EXAMPLE H

### Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

1. Use of one or more of the compounds of the formula I 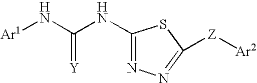
in which
Ar¹ denotes phenyl, naphthyl, biphenyl or Het, each of which is unsubstituted or mono-, di-, tri-, tetra- or pentasubstituted by R¹,
Ar² denotes phenyl, naphthyl, biphenyl or Het, each of which is unsubstituted or mono-, di-, tri-, tetra- or pentasubstituted by R²,
Y denotes O, S, CH—NO₂, C(CN)₂ or N—R⁴,
Z denotes —O—, —S—, —CH₂—(CH₂)ₙ—, —(CH₂)ₙ—CHA-, —CHA-(CH₂)ₙ—, —C(═O)—, —CH(OH)—, —(CHA)ₙO—, —(CH₂)ₙO—, —O(CHA)ₙ-, —O(CH₂)ₙ—, —(CH₂)ₙS—, —S(CH₂)ₙ—, —(CH₂)ₙNH—, —NH(CH₂)ₙ—, —(CH₂)ₙNA-, —NA(CH₂)ₙ—, —CHHal- or —C(Hal)₂-,
Het denotes a mono- or bicyclic aromatic heterocycle having 1 to 4 N, O and/or S atoms,
R¹, R², independently of one another, denote A, Ar′, OR³, SR³, OAr′, SAr′, N(R³)₂, NHAr′, Hal, NO₂, CN, (CH₂)ₙCOOR³, (CH₂)ₙCON(R³)ₙ, COR³, S(O)ₘA, S(O)ₘAr′, NHCOA, NHCOAr′, NHSOₘA, NHSOOmAr′, SOₘN(R³)₂, —O—(CH₂)ₙ—N(R³)₂, O(CH₂)ₙNHR³, O(CH₂)ₙNA₂, O(CH₂)ₙC(CH₃)₂(CH₂)ₙN(R³)₂, NH(CH₂)ₙ(CH₃)₂(CH₂)ₙN(R³)₂, O(CH₂)ₙN(R³)SOₘA, O(CH₂)ₙN(R³)SOₘN(R³)A, O(CH₂)ₙN(R³)SOₘAr′, (CH₂)ₙN(R³)SOₘA, (CH₂)ₙN(R³)SOₘN(R³)A, (CH₂)ₙN(R³)SOₘAr′, O(CH₂)ₙSOₘA, O(CH₂)ₙSOₘN(R³)A, O(CH₂)ₙSOₘAr′, (CH₂)ₙSOₘA, (CH₂)ₙSOₘN(R³)A, (CH₂)ₙSOₘAr′, —NH—(CH₂)ₙ—NH₂, —NH—(CH₂)ₙ—NHA, —NH—(CH₂)ₙ—NA₂, —NA-(CH₂)ₙ—NH₂, —NA-(CH₂)ₙ—NHA, —NA-(CH₂)ₙ—NA₂, —O—(CH₂)ₙ-Het¹ or Het¹,
R³ denotes H, A or (CH₂)ₙAr′,
R⁴ denotes H, CN, OH, A, (CH₂)ₘAr′, COR³, COAr′, S(O)ₘA or S(O)ₘAr′,
Ar′ denotes phenyl which is unsubstituted or mono-, di-, tri-, tetra- or pentasubstituted by A, Ph, OH, OA, SH, SA, OPh, SPh, NH₂, NHA, NA₂, NHPh, Hal, NO₂, CN, (CH₂)ₙCOOH, (CH₂)ₙCOOA, (CH₂)ₙCONH₂, (CH₂)ₙCONHA, CHO, COA, S(O)ₘA, S(O)ₘPh, NHCOA, NHCOPh, NHSO₂A, NHSO₂Ph or SO₂NH₂,
Ph denotes phenyl which is unsubstituted or mono-, di- or trisubstituted by A, Hal, CN, COOR, COOH, NH2, NO₂, OH or OA,
Het¹ denotes a monocyclic saturated heterocycle having 1 to 4 N, O and/or S atoms, which may be unsubstituted or mono-, di- or trisubstituted by Hal, A, OA, CN, (CH₂)ₙOH, (CH₂)ₙHal, NH₂, ═NH, ═N—OH, ═N—OA and/or carbonyl oxygen (═O),
A denotes alkyl having 1 to 10 C atoms, where 1-7 H atoms may also be replaced by F and/or chlorine,
Hal denotes F, Cl, Br or I,
n denotes 0, 1, 2, 3, 4 or 5,
m denotes 0, 1 or 2,
and pharmaceutically usable derivatives, solvates, salts and stereoisomers thereof, including mixtures thereof in all ratios, for the preparation of a medicament for the prophylaxis and/or treatment of diseases in which the inhibition, regulation and/or modulation of kinase signal transduction plays a role.

2. Use according to claim 1, characterised in that the disease is (are) caused, mediated and/or propagated by thyrosine and/or Raf kinase(s).

3. Use according to claim 2, characterised in that the disease is caused, mediated and/or propagated by A-Raf, B-Raf and/or Raf-1 kinase.

4. Use according to claim 1, characterised in that the disease is a hyperproliferative disease.

5. Use according to claim 4, characterised in that the disease is a cancer-like disease.

6. Use according to claim 5, characterised in that the disease is brain cancer, lung cancer, squamous epithelium cancer, bladder cancer, stomach cancer, pancreatic cancer, liver cancer, kidney cancer, colorectal cancer, breast cancer, head cancer, neck cancer, oesophageal cancer, gynaecological cancer, thyroid cancer, lymphoma, chronic leukaemia or acute leukaemia.

7. Use according to claim 4, characterised in that the disease is not cancer-like.

8. Use according to claim 7, characterised in that the disease is psoriasis, endometriosis, scarring or benign prostate hyperplasia.

9. Use according to claim 1, characterised in that the disease is an inflammation, arthritis, Helicobacter pylori infection, influenza A, an immunological disease, an autoimmune disease or an immunodeficiency disease.

10. Use according to claim 1, characterised in that a compound of the formula I is employed in which
Z denotes —CH₂—(CH₂)ₙ—, —(CH₂)ₙ—CHA, —CHA-O— or —O—,
and pharmaceutically usable derivatives, solvates, salts and stereoisomers thereof, including mixtures thereof in all ratios.

11. Compounds general formula VI 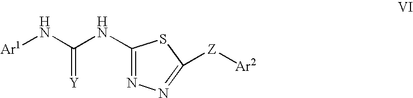
in which
Ar¹ denotes phenyl which is unsubstituted or mono-, di-, tri-, tetra- or pentasubstituted by R¹,
Ar² denotes phenyl or Het, each of which is unsubstituted or mono-, di-, tri-, tetra- or pentasubstituted by R²,
Y denotes O,
Z denotes —O—, —CH₂—(CH₂)ₙ—, —(CH₂)ₙ—CHA-, —CHA-(CH₂)ₙ—, —C(═O)—, —CH(OH)—, —CH(OA)-, —(CH₂)ₙO—, —O(CH₂)ₙ—, —(CH₂)ₙNH— or —NH(CH₂)ₙ—,
Het denotes a mono- or bicyclic aromatic heterocycle having 1 to 4 N, O and/or S atoms,
R¹, R², independently of one another, denote A, OR³, Hal, NO₂, CN, S(O)ₘA, O(CH₂)ₙNA₂ or Het¹,
R³ denotes H or A,
Het¹ denotes a monocyclic saturated heterocycle having 1 to 4 N, O and/or S atoms, which may be unsubstituted or mono-, di- or trisubstituted by Hal, A, OA, CN, (CH₂)ₙOH, (CH₂)ₙHal, NH₂, ═NH, ═N—OH, ═N—OA and/or carbonyl oxygen (═O),
A denotes alkyl having 1 to 10 C atoms, where 1-7 H atoms may also be replaced by F and/or chlorine,
Hal denotes F, Cl, Br or I,
n denotes 0, 1, or 2,
m denotes 0, 1 or 2,
and pharmaceutically usable derivatives, solvates, salts and stereoisomers thereof, including mixtures thereof in all ratios.

12. Compounds of the formula I according to claim 1, characterised in that these have the following structures:
1-(2-methoxy-5-trifluoromethylphenyl)-3-(5-pyridin-4-ylmethyl-1,3,4-thiadiazol-2-yl)urea,
1-(5-chloro-2-methoxy-4-methylphenyl)-3-[5-(3,4-dimethoxybenzyl)-1,3,4-thiadiazol-2-yl]urea,
1-[5-(3,4-dimethoxybenzyl)-1,3,4-thiadiazol-2-yl]-3-(3-trifluoromethoxyphenyl)urea,
1-[5-(1-phenylethyl)-1,3,4-thiadiazol-2-yl]-3-(3-trifluoromethanesulfonylphenyl)urea,
1-[5-(3,4-dimethoxybenzyl)-1,3,4-thiadiazol-2-yl]-3-(2-methoxy-5-trifluoromethylphenyl)urea,
1-[5-(1-phenylethyl)-1,3,4-thiadiazol-2-yl]-3-p-tolylurea,
1-(2-methoxy-5-methylphenyl)-3-[5-(1-phenylethyl)-1,3,4-thiadiazol-2-yl]urea,
1-(3-chloro-4-methylphenyl)-3-[5-(1-phenylethyl)-1,3,4-thiadiazol-2-yl]urea,
1-(5-chloro-2-methylphenyl)-3-[5-(1-phenylethyl)-1,3,4-thiadiazol-2-yl]urea,
1-(3-chloro-2-methylphenyl)-3-[5-(1-phenylethyl)-1,3,4-thiadiazol-2-yl]urea,
1-(5-chloro-2-methoxyphenyl)-3-[5-(1-phenylethyl)-1,3,4-thiadiazol-2-yl]urea,
1-[5-(1-phenylethyl)-1,3,4-thiadiazol-2-yl]-3-(3-trifluoromethylphenyl)-urea,
1-[5-(1-phenylethyl)-1,3,4-thiadiazol-2-yl]-3-(4-trifluoromethylphenyl)-urea,
1-[5-(3,4-dimethoxybenzyl)-1,3,4-thiadiazol-2-yl]-3-(2-methoxyphenyl)-urea,
1-[5-(1-phenylethyl)-1,3,4-thiadiazol-2-yl]-3-(4-trifluoromethoxyphenyl)urea,
1-(4-fluoro-3-trifluoromethylphenyl)-3-[5-(1-phenylethyl)-1,3,4-thiadiazol-2-yl]urea,
1-(4-chloro-3-trifluoromethylphenyl)-3-[5-(1-phenylethyl)-1,3,4-thiadiazol-2-yl]urea,
1-[5-(2,3-dimethoxybenzyl)-1,3,4-thiadiazol-2-yl]-3-(4-trifluoromethoxyphenyl)urea,
1-[5-(2,3-dimethoxybenzyl)-1,3,4-thiadiazol-2-yl]-3-(2-trifluoromethoxyphenyl)urea,
1-(5-chloro-2,4-dimethoxyphenyl)-3-[5-(3,4-dimethoxybenzyl)-1,3,4-thiadiazol-2-yl]urea,
1-(2,4-dimethoxyphenyl)-3-[5-(1-phenylethyl)-1,3,4-thiadiazol-2-yl]urea,
1-(3-chloro-4-methoxyphenyl)-3-[5-(1-phenylethyl)-1,3,4-thiadiazol-2-yl]urea,
1-[2-(2-dimethylaminoethoxy)-5-trifluoromethylphenyl]-3-[5-(1-phenylethyl)-1,3,4-thiadiazol-2-yl]urea,
1-[4-chloro-5-methyl-2-(piperidin-4-yloxy)phenyl]-3-[5-(3,4-dimethoxybenzyl)-1,3,4-thiadiazol-2-yl]urea,
1-(2-methoxy-5-trifluoromethylphenyl)-3-[5-(1-phenylethyl)-1,3,4-thiadiazol-2-yl]urea,
1-(5-chloro-2-methoxy-4-methylphenyl)-3-(5-pyridin-4-ylmethyl-1,3,4-thiadiazol-2-yl)urea,
1-(5-pyridin-4-ylmethyl-1,3,4-thiadiazol-2-yl)-3-(3-trifluoromethoxyphenyl)urea,
1-(5-chloro-2-methoxy-4-methylphenyl)-3-[5-(1-phenylethyl)-1,3,4-thiadiazol-2-yl]urea,
1-[5-(1-phenylethyl)-1,3,4-thiadiazol-2-yl]-3-(3-trifluoromethoxy-7-phenyl)urea,
1-(2-methoxy-5-trifluoromethylphenyl)-3-[5-(1-phenylpropyl)-1,3,4-thiadiazol-2-yl]urea,
1-(5-chloro-2-methoxy-4-methylphenyl)-3-[5-(4-chlorophenoxymethyl)-1,3,4-thiadiazol-2-yl]urea,
1-[5-(4-chlorophenoxymethyl)-1,3,4-thiadiazol-2-yl]-3-(3-trifluoromethoxyphenyl)urea,
1-[4-chloro-2-(2-dimethylaminoethoxy)-5-methylphenyl]-3-[5-(1-phenylethyl)-1,3,4-thiadiazol-2-yl]urea,
1-[4-chloro-2-(2-dimethylaminoethoxy)-5-methylphenyl]-3-[5-(3,4-dimethoxybenzyl)-1,3,4-thiadiazol-2-yl]urea,
1-[5-(3,4-dimethoxybenzyl)-1,3,4-thiadiazol-2-yl]-3-[2-(2-dimethylaminoethoxy)-5-trifluoromethylphenyl]urea,
1-(2-methoxy-5-methylphenyl)-3-[5-(1-phenylpropyl)-1,3,4-thiadiazol-2-yl]urea,
1-(2,5-dimethoxyphenyl)-3-[5-(1-phenylethyl)-1,3,4-thiadiazol-2-yl]urea,
1-(2,5-dichlorophenyl)-3-[5-(1-phenylethyl)-1,3,4-thiadiazol-2-yl]urea,
1-[5-(hydroxyphenylmethyl)-1,3,4-thiadiazol-2-yl]-3-(3-trifluoromethylphenyl)urea,
1-(2-methoxy-5-methylphenyl)-3-[5-(2-methyl-1-phenylpropyl)-1,3,4-thiadiazol-2-yl]urea,
1-(2-fluoro-5-trifluoromethylphenyl)-3-(5-pyridin-4-ylmethyl-1,3,4-thiadiazol-2-yl)urea,
1-(4-fluoro-3-trifluoromethylphenyl)-3-(5-pyridin-4-ylmethyl-1,3,4-thiadiazol-2-yl)urea,
1-[5-(3,4-dimethoxybenzoyl)-1,3,4-thiadiazol-2-yl]-3-m-tolylurea,
1-{5-[2-(3,4-dimethoxyphenyl)ethyl]-1,3,4-thiadiazol-2-yl}-3-m-tolylurea,
1-(3-chloro-4-methylphenyl)-3-[5-(2-methyl-1-phenylpropyl)-1,3,4-thiadiazol-2-yl]urea,
1-(3-chlorophenyl)-3-[5-(3,4-dimethoxyphenoxy)-1,3,4-thiadiazol-2-yl]urea,
1-(3-chlorophenyl)-3-[5-(3,4-dimethoxybenzoyl)-1,3,4-thiadiazol-2-yl]urea,
1-(3-chlorophenyl)-3-{5-[2-(3,4-dimethoxyphenyl)ethyl]-1,3,4-thiadiazol-2-yl}urea,
1-(5-chloro-2,4-dimethoxyphenyl)-3-[5-(1-phenylethyl)-1,3,4-thiadiazol-2-yl]urea,
1-(3-chlorophenyl)-3-[5-(3,4-dimethoxybenzylamino)-1,3,4-thiadiazol-2-yl]urea,
1-[5-(3,4-dimethoxyphenylamino)-1,3,4-thiadiazol-2-yl]-3-(3-trifluoromethylphenyl)urea,
1-[5-(3,4-dimethoxyphenoxy)-1,3,4-thiadiazol-2-yl]-3-(3-trifluoromethylphenyl)urea,
1-[5-(4-chlorophenoxymethyl)-1,3,4-thiadiazol-2-yl]-3-(4-fluoro-3-trifluoromethylphenyl)urea,
1-(5-chloro-2-methoxyphenyl)-3-{5-[2-(3,4-dimethoxyphenyl)ethyl]-1,3,4-thiadiazol-2-yl}urea,
1-(5-chloro-2-methoxyphenyl)-3-[5-(3,4-dimethoxybenzoyl)-1,3,4-thiadiazol-2-yl]urea,
1-(5-chloro-2-methoxyphenyl)-3-[5-(3,4-dimethoxybenzylamino)-1,3,4-thiadiazol-2-yl]urea,
1-{5-[2-(3,4-dimethoxyphenyl)ethyl]-1,3,4-thiadiazol-2-yl}-3-(3-trifluoromethylphenyl)urea,
1-[5-(3,4-dimethoxybenzylamino)-1,3,4-thiadiazol-2-yl]-3-(3-trifluoromethylphenyl)urea,
1-[5-(3,4-dimethoxyphenylamino)-1,3,4-thiadiazol-2-yl]-3-(2-fluoro-3-trifluoromethylphenyl)urea,
1-[5-(3,4-dimethoxyphenoxy)-1,3,4-thiadiazol-2-yl]-3-(2-fluoro-3-trifluoromethylphenyl)urea,
1-[5-(3,4-dimethoxyphenoxy)-1,3,4-thiadiazol-2-yl]-3-(4-fluoro-3-trifluoromethylphenyl)urea,
1-[5-(3,4-dimethoxybenzoyl)-1,3,4-thiadiazol-2-yl]-3-(3-fluoro-5-trifluoromethylphenyl)urea,
1-{5-[2-(3,4-dimethoxyphenyl)ethyl]-1,3,4-thiadiazol-2-yl}-3-(3-fluoro-5-trifluoromethylphenyl)urea,
1-[5-(3,4-dimethoxybenzoyl)-1,3,4-thiadiazol-2-yl]-3-(2-fluoro-5-trifluoromethylphenyl)urea,
1-[5-(3,4-dimethoxybenzoyl)-1,3,4-thiadiazol-2-yl]-3-(4-fluoro-3-trifluoromethylphenyl)urea,
1-[5-(3,4-dimethoxybenzoyl)-1,3,4-thiadiazol-2-yl]-3-(2-fluoro-3-trifluoromethylphenyl)urea,
1-{5-[2-(3,4-dimethoxyphenyl)ethyl]-1,3,4-thiadiazol-2-yl}-3-(4-fluoro-3-trifluoromethylphenyl)urea,
1-{5-[2-(3,4-dimethoxyphenyl)ethyl]-1,3,4-thiadiazol-2-yl}-3-(2-fluoro-3-trifluoromethylphenyl)urea,
1-{5-[2-(3,4-dimethoxyphenyl)ethyl]-1,3,4-thiadiazol-2-yl}-3-(2-fluoro-5-trifluoromethylphenyl)urea,
1-[5-(3,4-dimethoxybenzylamino)-1,3,4-thiadiazol-2-yl]-3-(2-fluoro-3-trifluoromethylphenyl)urea,
1-(4-chloro-3-trifluoromethylphenyl)-3-{5-[2-(3,4-dimethoxyphenyl)ethyl]-1,3,4-thiadiazol-2-yl)}urea,
1-(4-chloro-3-trifluoromethylphenyl)-3-[5-(3,4-dimethoxybenzoyl)-1,3,4-thiadiazol-2-yl]urea,
1-(4-chloro-3-trifluoromethylphenyl)-3-[5-(3,4-dimethoxybenzylamino)-1,3,4-thiadiazol-2-yl]urea,
1-(3,5-bistrifluoromethylphenyl)-3-[5-(3,4-dimethoxyphenylamino)-1,3,4-thiadiazol-2-yl]urea,
1-(3,5-bistrifluoromethylphenyl)-3-[5-(3,4-dimethoxybenzoyl)-1,3,4-thiadiazol-2-yl]urea,
1-(3,5-bistrifluoromethylphenyl)-3-{5-[2-(3,4-dimethoxyphenyl)ethyl]-1,3,4-thiadiazol-2-yl}urea,
1-(3,5-bistrifluoromethylphenyl)-3-[5-(3,4-dimethoxybenzylamino)-1,3,4-thiadiazol-2-yl]urea,
1-(3-chlorophenyl)-3-[5-(pyridin-4-yloxy)-1,3,4-thiadiazol-2-yl]urea,
1-[5-(pyridin-4-yloxy)-1,3,4-thiadiazol-2-yl]-3-(3-trifluoromethylphenyl)urea,
1-(4-fluoro-3-trifluoromethylphenyl)-3-[5-(pyridin-4-yloxy)-1,3,4-thiadiazol-2-yl]urea,
1-(2-fluoro-3-trifluoromethylphenyl)-3-[5-(pyridin-4-yloxy)-1,3,4-thiadiazol-2-yl]urea,
1-(2-fluoro-5-trifluoromethylphenyl)-3-[5-(pyridin-4-yloxy)-1,3,4-thiadiazol-2-yl]urea,
1-(3,5-bistrifluoromethylphenyl)-3-[5-(pyridin-4-yloxy)-1,3,4-thiadiazol-2-yl]urea,
1-(5-chloro-2-methoxyphenyl)-3-[5-(4-chlorophenoxymethyl)-1,3,4-thiadiazol-2-yl]urea,
1-[5-(4-chlorophenoxymethyl)-1,3,4-thiadiazol-2-yl]-3-(3-trifluoromethylphenyl)urea,
1-[5-(3,4-dimethoxybenzoyl)-1,3,4-thiadiazol-2-yl]-3-(3-trifluoromethylphenyl)-urea,
1-[5-(3,4-dimethoxyphenoxymethyl)-1,3,4-thiadiazol-2-yl]-3-m-tolylurea,
1-(3-chlorophenyl)-3-[5-(3,4-dimethoxyphenoxymethyl)-1,3,4-thiadiazol-2-yl]urea,
1-(5-chloro-2-methoxyphenyl)-3-[5-(3,4-dimethoxyphenoxymethyl)-1,3,4-thiadiazol-2-yl]urea,
1-[5-(3,4-dimethoxyphenoxymethyl)-1,3,4-thiadiazol-2-yl]-3-(3-trifluoromethylphenyl)urea,
1-[5-(3,4-dimethoxyphenoxymethyl)-1,3,4-thiadiazol-2-yl]-3-(2-fluoro-3-trifluoromethylphenyl)urea,
1-[5-(3,4-dimethoxyphenoxymethyl)-1,3,4-thiadiazol-2-yl]-3-(3-fluoro-5-trifluoromethylphenyl)urea,
1-[5-(3,4-dimethoxyphenoxymethyl)-1,3,4-thiadiazol-2-yl]-3-(4-fluoro-3-trifluoromethylphenyl)urea,
1-[5-(3,4-dimethoxyphenoxymethyl)-1,3,4-thiadiazol-2-yl]-3-(2-fluoro-5-trifluoromethylphenyl)urea,
1-(4-chloro-3-trifluoromethylphenyl)-3-[5-(3,4-dimethoxyphenoxymethyl)-1,3,4-thiadiazol-2-yl]urea,
1-(3,5-bistrifluoromethylphenyl)-3-[5-(3,4-dimethoxyphenoxymethyl)-1,3,4-thiadiazol-2-yl]urea,
(S)-1-[5-(1-phenylethyl)-1,3,4-thiadiazol-2-yl]-3-(3-trifluoromethylphenyl)urea,
(R)-1-[5-(1-phenylethyl)-1,3,4-thiadiazol-2-yl]-3-(3-trifluoromethylphenyl)urea,
(S)-1-(5-chloro-2-methoxyphenyl)-3-[5-(1-phenylethyl)-1,3,4-thiadiazol-2-yl]urea enantiomer,
(R)-1-(5-chloro-2-methoxyphenyl)-3-[5-(1-phenylethyl)-1,3,4-thiadiazol-2-yl]urea,
(S)-1-(4-fluoro-3-trifluoromethylphenyl)-3-[5-(1-phenylethyl)-1,3,4-thiadiazol-2-yl]urea,
(R)-1-(4-fluoro-3-trifluoromethylphenyl)-3-[5-(1-phenylethyl)-1,3,4-thiadiazol-2-yl]urea
and pharmaceutically usable derivatives, solvates, salts and stereoisomers thereof, including mixtures thereof in all ratios.

13. Process for the preparation of the compounds according to claim 11 and pharmaceutically usable derivatives, salts, solvates and stereoisomers thereof, characterised in that
a) a compound of the formula II 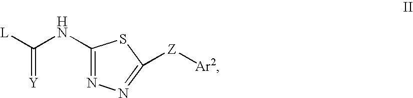
in which Y, Z and Ar² each have the same meaning as in the compound according to claim 11 to be prepared,
and L denotes Cl, Br, I or a free or reactively functionally modified OH group,
is reacted with a compound of the formula III
Ar¹—NH₂ III,
in which Ar¹ has the same meaning as in the compound according to claim 11 to be prepared,
or
b) a compound of the formula IV 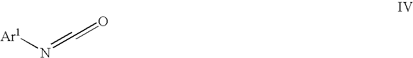
in which Ar¹ has the same meaning as in the compound according to claim 11 to be prepared,
is reacted with a compound of the formula V 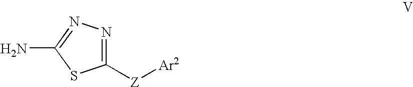
in which Z and Ar² each have the same meaning as in the compound according to claim 11 to be prepared,
and/or
a base or acid of the formula I is converted into one of its salts.

14. Medicament comprising at least one compound according to claim 11 and/or one of its pharmaceutically usable derivatives, salts, solvates and stereoisomers, including mixtures thereof in all ratios, and optionally excipients and/or adjuvants.

15. Set (kit) consisting of separate packs of
a) an effective amount of a compound of the formula I and/or pharmaceutically usable derivatives, solvates and stereoisomers thereof, including mixtures thereof in all ratios,
and
b) an effective amount of a further medicament active ingredient.
